(19) United States
(12) Patent Application Publication
(10) Pub. No.: US 2026/0271926 A1
(43) Pub. Date: Sep. 17, 2026

(54) BACTERIA-RESPONSIVE PARTICLES, METHODS FOR PREPARATION, AND USE IN PACKAGING OR ON SURFACES

(71) Applicants: Emmanuel Abraham HO, Waterloo (CA); Chuying FENG, Toronto (CA); Ethan Joseph WATT, Guelph-Eramosa (CA)

(72) Inventors: Emmanuel Abraham HO, Waterloo (CA); Chuying FENG, Toronto (CA); Ethan Joseph WATT, Guelph-Eramosa (CA)

(21) Appl. No.: 19/469,495

(22) PCT Filed: Mar. 27, 2024
(86) PCT No.: PCT/CA2024/050386

§ 371 Date: Sep. 26, 2025

Related U.S. Application Data
(60) Provisional application No. 63/577,061, filed on Mar. 30, 2023.

(51) Int. Cl. A01N 37/46; A01N 25/28; A01N 25/34; A01P 1/00; B82Y 5/00

(52) U.S. Cl. CPC A01N 37/46; A01N 25/28; A01N 25/34; A01P 1/00; B82Y 5/00

(57) ABSTRACT

Bacteria-responsive particles and a platform for product packaging or for incorporating on surfaces are described, together with uses thereof and methods of preparation. The particles, such as liposomes or lipid nanoparticles, provide a bacteria-responsive platform for release of an agent, which may be antimicrobial, only in the presence of the target bacteria upon release of virulence factors referred to as cholesterol-dependent cytolysins (CDCs) by the bacteria. Common bacteria involved in food poisoning, such as bacillus, Clostridium, listeria, and streptococcus, produce CDCs and can be targeted with the particles described. The particles are cholesterol-rich, and comprise surface-exposed cholesterol. The releasable agent is encapsulated within the particles. Interaction of the surface-exposed cholesterol with the CDC released from the target bacteria causes particle porosity and release of the agent encapsulated within the particles, permitting the agent to take effect, such as to deter the target bacteria or indicate its presence.

### CROSS REFERENCE TO RELATED APPLICATIONS

This application claims the benefit of and priority to U.S. Provisional Patent Application No. 63/577,061 filed Mar. 30, 2023 and entitled “BACTERIA-RESPONSIVE DRUG RELEASE PLATFORM FOR FOOD PACKAGING”, the contents of which are expressly incorporated by reference herein for all purposes.

### FIELD

The present disclosure relates to particles such as liposomes or nanoparticles, methods of preparation, and uses thereof for packaging of products, such as food products, and for application to surfaces so as to deter bacterial growth.

### BACKGROUND

A 2023 United Nations Report estimates that 17% of the total global food production is wasted which is due in part to the short shelf-life of food products [1]. Preservatives are used to prevent spoilage, improve appearance, and maintain nutritional quality of a food. However, preservatives only prolong the shelf life of a food, and do not eliminate bacteria. By the time bacteria growth is detected or evident it is too late, and the spoiled food product must be discarded. Further, certain preservatives may have unwanted effects, or may be deemed unacceptable by consumers. Designing improved food packaging can help prolong the shelf-life and can dramatically reduce food spoilage, in the interest of enhancing food sustainability and global food security. Other consumer products with a limited shelf-life, or which may be at risk of spoilage such as within the drug, cosmetic, oral hygiene or feminine hygiene industries could benefit from packaging enhancements that extend shelf-life.

Previous technologies for food-borne pathogen detection have been proposed, such as for food spoilage detection. Such technologies may involve detection of a change in pH as indicative of the presence of bacterial growth, for example as taught in US 2004/0115319 A1. Sensors for detecting a pH change indicative of microbial growth based on a change in a polymeric material are described, for example, in U.S. Pat. No. 10,782,238 B2.

Antimicrobial nanofilms have been proposed, which may be based upon inclusion of titanium nano-dioxide within the film, as described in US U.S. Pat. No. 11,306,186 B2; or as described in US 2020/0205403 A1 where the antimicrobial film combines nano-titanium dioxide with nano-silver and nano-clay particles.

Lipid particles, such as lipid nanoparticles (LNPs) have been used for delivery of active molecules, such as short interfering nucleic acid (siNA) molecules, as described in US 2008/0020058 A1. Lipid nanoparticles, such as particles having a single phospholipid bilayer, are well known. Peptides, such as lysin polypeptides which may be active against certain bacteria, are taught for example in WO 2017/049233. Apart from the food industry, cosmetic-grade anti-bacterial agents and formulation for their delivery are known and taught. For example in WO 2015/114666 pertains to treatment of acne with anti-bacterial agents.

It is desirable to find alternatives to the previous technologies so as to obviate or mediate one or more shortcomings of previous technologies aimed at reducing microbial growth within consumer products.

### SUMMARY

Described herein are bacteria-responsive particles encapsulating a releasable agent for interaction with a target bacteria that releases a cholesterol-dependent cytolysin (CDC), said particles comprising: surface-exposed cholesterol, and said releasable agent encapsulated within the particles, for interaction with the target bacteria upon release, wherein interaction of the surface-exposed cholesterol of the particles with the CDC released from the target bacteria causes particle porosity and release of the agent from the particles.

A film or coating comprising the bacteria-responsive particles is also described. A food packaging container or food packaging insert comprising the bacteria-responsive particles is described herein. Further, a substrate or surface comprising the bacteria-responsive particles is described, wherein the substrate or surface comprises packaging for a food, cosmetic, pharmaceutical, hygiene product packaging, a medical device, or a substrate or surface susceptible to growth of, or exposure to, the target bacteria.

Further, described herein is a method of preparing bacteria-responsive particles encapsulating a releasable agent for interaction with a target bacteria when the target bacteria releases a cholesterol-dependent cytolysin (CDC). The method comprises: preparing a particle-forming composition for providing particles with a lipid-rich total cholesterol content of from 30-85 mol %, from 55-75 mol %, or from 60-70 mol %, said composition comprising the releasable agent; sonicating the composition for 80 s to 600 s, such as about 200 s; forming particles having an average diameter of from 50 to 1000 nm, an average diameter of from 100 nm to 500 nm, an average diameter of from 300 to 400 nm, or having an average diameter of about 300 nm; filtering the particles to exclude sizes over about 35 kDa, over about 30 kDa, or over about 20 kDa; and washing the particles to remove non-encapsulated agent; wherein the prepared particles comprise surface-exposed cholesterol, and the releasable agent is encapsuled within said particles.

Particles are described herein for use as a bacteria-responsive platform for release of one or more agent, such as an antimicrobial agent, in the presence of specific target bacteria. The bacteria-responsive particles, such as liposomes or lipid nanoparticles, releases the agent only in the presence of specific bacteria. Common bacteria involved in food poisoning, such as bacillus, clostridium, listeria, and streptococcus, produce a class of virulence factors called cholesterol-dependent cytolysins. (CDCs). The nanotechnology-based formulation is in the form of a particle that achieves sustained release of an active ingredient over an extended period of days in response to CDC production by the bacteria to be addressed. Absent the bacteria to be addressed, the agent is not released, but remains contained within the particle.

The particles described herein provide a smart, on-demand bacteria-responsive release of the intended agent that can specifically deter bacterial growth. The particles and formulations comprising the particles may be used for smart food packaging. By reducing the exposure of consumer products to microbes, the exposure and risk to the consumer is reduced, not only to bacteria, but to unwanted preservatives or chemicals conventionally used within a food to prolong the shelf-life of food products.

Advantageously, the particles, and their use in a formulation and/or platform, show high specificity of release of the active agent, such as an anti-microbial agent, in response to bacteria that produce CDCs. Thus, the shelf-life of food products can be prolonged. This can reduce the use of food preservatives within the food product which may be unappealing to the consumer, or which may have unwanted side effects. The agent released from the formulation may advantageously reduce or eliminate the bacteria responsible for food or product spoilage.

Other aspects and features of the present disclosure will become apparent to those ordinarily skilled in the art upon review of the following description of specific embodiments in conjunction with the accompanying figures.

### BRIEF DESCRIPTION OF THE FIGURES

Embodiments of the present disclosure will now be described, by way of example only, with reference to the attached Figures.

FIG. 1 is a schematic representation of a particle, which is a liposome, formed as described herein, for release of an antimicrobial agent in response to release of CDCs by bacteria.

FIG. 2 shows a calcein calibration curve.

FIG. 3 illustrates a fluorescence spectra (500-650 nm) of calcein aqueous solutions with different concentration ranging from 0.1 μg/mL to 4 μg/mL.

FIG. 4 shows the Z-average diameter values are the means of six repeat measurements of liposomes prepared by sonication.

FIG. 5 illustrates the encapsulation efficiency (%) of liposome formulations.

FIG. 6 shows the size distribution of liposomes with varying sonication time lengths from 80 s to 600 s.

FIG. 7 shows G. vaginalis growth (OD₆₀₀) in liquid culture, with 100 μL cells inoculated into 10 mL broth and incubated at 37° C.

FIG. 8 shows a Columbia agar plate with no bacteria growth observed after one week.

FIG. 9 shows a 1685 NYC III agar plate (spreading), with a bacteria lawn formed after 48 hours.

FIG. 10 shows 1685 NYC III agar plate (streaking), with few CFUs after 48 hours.

FIG. 11 shows Casman agar plate (streaking), showing CFUs found after 36-48 hours.

FIG. 12 shows CFUs of G. vaginalis grown on Casman agar plates, as follows: Panel a) 1,000-fold dilution; Panel b) 10,000-fold dilution; and Panel c) 100,000-fold dilution.

FIG. 13 shows L. crispatus growth in liquid culture.

FIG. 14 shows CFUs of L. crispatus grown on MRS agar plates, as follows:

Panel a) 100-fold dilution; Panel b) 1,000-fold dilution; Panel c) 10,000-fold dilution; and Panel d) 100,000-fold dilution.

FIG. 15 shows a calcein calibration curve in 1685 NYC III medium.

FIG. 16 shows a calcein calibration curve in MRS medium.

FIG. 17 shows calcein fluorescence stability in 1685 NYC III medium and G. vaginalis supernatant.

FIG. 18 pertains to Formulation 1 of Example 1, showing cumulative calcein release stimulated by G. vaginalis.

FIG. 19 relates to Formulation 2 of Example 1, showing cumulative calcein release stimulated by G. vaginalis.

FIG. 20 pertains to Formulation 3 of Example 1, showing cumulative calcein release stimulated by G. vaginalis.

FIG. 21 relates to Formulation 4 of Example 1, showing cumulative calcein release stimulated by G. vaginalis.

FIG. 22 shows a comparison of cumulative calcein release stimulated by G. vaginalis.

FIG. 23 shows cumulative calcein released from Formulation 2 in the presence of G. vaginalis supernatant or L. crispatus supernatant.

FIG. 24 depicts a microfluidic system for preparing liposomes according to Example 2.

FIG. 25 provides contour plots in Panels (a) to (I), highlighting the influence and interplay between different independent variables: nisin, FRR and PG.

FIG. 26 shows the impact of cholesterol content on liposomal properties, in Panel (a) the PS and PDI, and in Panel (b) the EE and LC.

FIG. 27 shows hemolytic activity of purified PLY toxin and bacterial culture Panels (a)-(b) against human erythrocytes; and sequestration capabilities of liposomes with varying cholesterol content against purified PLY and bacterial culture Panels (c)-(d).

FIG. 28 shows liposomal release of nisin Z via PLY interactions after incubation at 37° C. for a total of 1 hr at varying molar compositions of cholesterol.

FIG. 29 shows absorbance readings at 600 nm to indicate turbidity of bacterial solution with varying nisin concentration.

FIG. 30 illustrates liposomal films of varying molarity. Panel (a) indicates that films were affixed to the bottom of a 96 well plate and fully dried. Panel (b) shows the protective effect of the films in hemolytic assays of the treated wells at varying cholesterol content and molarity from 30 mol % to 75 mol % with PLY.

### DETAILED DESCRIPTION

Bacteria-responsive particles are described herein which encapsulate a releasable agent for interaction with a target bacteria that releases a cholesterol-dependent cytolysin (CDC). The particles comprise surface-exposed cholesterol, and the releasable agent is encapsulated within the particles, for interaction with the target bacteria upon release. Interaction of the surface-exposed cholesterol of the particles with the CDC released from the target bacteria causes particle porosity and release of the agent from the particles.

When the CDC binds to the surface-exposed cholesterol on the surface of the particles, the particle porosity is caused by pores forming in the surface of the particles, rendering the particles permeable to release the agent through the pores so formed.

In certain embodiments, the surface of the particles comprises a lipid bilayer or a phospholipid layer. For example, the particles may comprise liposomes, nanoparticles, lipid nanoparticles, polymeric nanoparticles or combinations thereof, together in a mixture.

The particles are cholesterol-rich, and may comprise from 30-80 mol % cholesterol, from 55-75 mol % cholesterol, or 60-70 mol % cholesterol.

Optionally, the particles may comprise, for example egg phosphatidylcholine (EPC), egg phosphatidylglycerol-sodium (EPG-Na), and/or propylene glycol among other components. Such other components may include a natural phospholipid sourced from egg or soybean, a synthetic phospholipid, a neutral phospholipid, an anionic phospholipid, a cationic phospholipid, a surface ligand, or an ionizable lipid. Exemplary components include but are not limited to: Phosphatidylcholine (PC); Phosphatidylethanolamine (PE); Phosphatidylserine (PS); Phosphatidylglycerol (PG); Phosphatidic acid (PA); Phosphatidylinositol (PI); Phosphatidylethanolamine (DPPE); Dipalmitoylphosphatidylcholine (DPPC); 1,2-distearoyl-sn-glycero-3-phosphoethanolamine (DSPE); 1,2-distearoyl-sn-glycero-3-phosphocholine (DSPC); 1,2-Dioleoylphosphatidylethanolamine (DOPE); 1,2-Dioleoyl-sn-glycero-3-phosphocholine (DOPC); Hydrogenated Soybean Phosphatidylcholine (HSPC); 1-palmitoyl-2-oleoyl-sn-glycero-3-phosphocholine (POPC); 1,2-Dimyristoyl-sn-glycero-3-phosphocholine (DMPC); 1,2-Dipalmitoyl-sn-glycero-3-phosphatidic acid sodium salt (DPPA); Dipalmitoylphosphatidylglycerol (DPPG); 1,2-Distearoyl-sn-glycero-3-phosphate (DSPA); 1,2-Distearoyl-sn-glycero-3-phosphoglycerol (DSPG); 1,2-dioleoyl-3-trimethylammonium-propane (DOTAP); 1,2-di-O-octadecenyl-3-trimethylammonium propane (DOTMA); DC-cholesterol (hydrochloride) (DC-Chol); Dimethyldioctadecylammonium (bromide) (DDAB); Polyethylene oxide (PEG); Hyaluronic acid (HA); Polyvinyl acetate (PVA); Chitosan; a protein; an antibody; an aptamer; (6Z,9Z,28Z,31Z)-Heptatriaconta-6,9,28,31-tetraen-19-yl 4-(dimethylamino)butanoate (DLin-MC3-DMA); 1,2-dioleoyl-3-dimethylammonium-propane (DODAP); [(4-hydroxybutyl)azanediyl]di(hexane-6,1-diyl)bis(2-hexyldecanoate) (ALC-0315); 9-Heptadecanyl 8-{(2-hydroxyethyl) [6-oxo-6-(undecyloxy) hexyl]amino}octanoate (SM-102); 1,2-dioleyloxy-3-dimethylaminopropane (DODMA); N-(4-carboxybenzyl)-N,N-dimethyl-2,3-bis(oleoyloxy) propan-1-aminium (DOBAQ); 1-methyl-4,4-bis[(9Z,12Z)-9,12-octadecadien-1-yloxy]-piperidine (YSK05); N,N-dimethyl-2,3-bis[(9Z,12Z)-9,12-octadecadien-1-yloxy]-1-propanamine (DLin-DMA); N,N-dimethyl-2,2-di-(9Z,12Z)-9,12-octadecadien-1-yl-1,3-dioxolane-4-ethanamine (DLin-KC2-DMA); 5-(dimethylamino)-pentanoic acid, (6Z)-1,2-di-(4Z)-4-decen-1-yl-6-dodecen-1-yl ester (CL1); 1,2-Distearoyl-sn-Glycero-3-Phosphatidylglycerol sodium (5A2-SC8); or a combination thereof, optionally together with any other compatible component.

The releasable agent may be an antibacterial agent, an indicator, a preservative, a bactericide, a broad spectrum bactericide, a fungicide, a virucide, an antimicrobial organic acid, a bacteriocin, a protein toxin, an antimicrobial peptide, an enzyme, an essential oil with bactericidal activity, a plant extract, a metal ion, an oxidizer, an antioxidant, or a combination thereof. Further, the releasable agent may be a food-grade, cosmetic-grade, or pharmaceutical-grade agent, depending on the intended application. For example, the releasable agent is an antimicrobial peptide (AMP), which may optionally present in the particles in an amount of from 10 to 200 μg/mL, or from 50 to 100 μg/mL.

The particles may have an average diameter of from 50 to 1000 nm, an average diameter of from 100 nm to 500 nm, an average diameter of from 300 to 400 nm, or having an average diameter of about 300 nm. The particle size distribution may be represented by a polydispersity index (PDI) value of from about 0.2 to 0.3, or from 0.25 to 0.28. Further, the particles may have a zeta potential of from −20 to −80 mV, from −25 to −70, or from −40 to −60 mV to render the particles stable through electrostatic repulsion.

The particles described herein may be for use in a system wherein the CDC and target bacteria are selected from amongst the following, but are not limited to: tetanolysin from C. tetani; botulinolysin from C. botulinum; perfringolysin O from C. perfringens; septicolysin O from C. septicum; histolyticolysin O from C. histolyticum; novyilysin from C. Novyi A (oedematiens); chauveolysin from C. chauvoei; bifermentolysin from C. bifermentans; sordellilysin from C. sordelli; pyolysin from A. pyogenes; laterosporolysin from B. laterosporus; vaginolysin from G. vaginalis; inerolysin from L. iners; aveolysin from P. alvei; lectinolysin from S. mitis; intermedilysin from S. intermedius; suilysin from S. suis; pneumolysin from S. pneumoniae; streptolysin O from S. canis; streptolysin O from S. Dysgalactiae subsp. Equisimilis; streptolysin O from S. pyogenes; cereolysin O from B. cereus; thuringolysin O from B. thuringiensis; anthrolysin O from B. anthracis; seeligerolysin O from L. seeligeri; ivanolysin O from L. ivanovii; and listeriolysin O from L. monocytogenes.

A film or coating comprising the bacteria-responsive particles described herein is one of the many applications of the particles described, which may be used in a wide variety of industries, such as for packaging.

A food packaging container or food packaging insert may comprise the bacteria-responsive particles described herein.

A substrate or surface comprising the bacteria-responsive particles described herein may be formed, or may have the particles applied thereto. Such a substrate or surface may be utilized for the packaging for a food, cosmetic, pharmaceutical, hygiene product packaging, a medical device, or a substrate or surface susceptible to growth of, or exposure to, the target bacteria.

A method is described herein form preparing bacteria-responsive particles encapsulating a releasable agent for interaction with a target bacteria, when the target bacteria releases a cholesterol-dependent cytolysin (CDC). The method comprises: preparing a particle-forming composition for providing particles with a lipid-rich total cholesterol content of from 30-85 mol %, from 55-75 mol %, or from 60-70 mol %, said composition comprising the releasable agent; sonicating the composition for 80 s to 600 s, such as about 200 s; forming particles having an average diameter of from 50 to 1000 nm, an average diameter of from 100 nm to 500 nm, an average diameter of from 300 to 400 nm, or having an average diameter of about 300 nm; filtering the particles to exclude sizes over about 35 kDa, over about 30 kDa, or over about 20 kDa; and washing the particles to remove non-encapsulated agent; wherein the prepared particles comprise surface-exposed cholesterol, and the releasable agent is encapsuled within the particles.

The particles may be liposomes, nanoparticles, lipid nanoparticles, polymeric nanoparticles or may be a combination thereof.

In an exemplary method, egg L-α-phosphatidylglycerol (EPG) and L-α-phosphatidylcholine (EPC) may be included in the particle-forming composition. Further, a wide choice of components may be included, such as natural phospholipid sourced from egg or soybean, a synthetic phospholipid, a neutral phospholipid, an anionic phospholipid, a cationic phospholipid, a surface ligand, an ionizable lipid, or a combination thereof, any of which may be included in the particle-forming composition for use in the described method. Exemplary components, such as described above in respect of the particles, may be used in the particle forming composition.

In the described method, the releasable agent may be an antibacterial agent, an indicator, a preservative, a bactericide, a broad spectrum bactericide, a fungicide, a virucide, an antimicrobial organic acid, a bacteriocin, a protein toxin, an antimicrobial peptide, an enzyme, an essential oil with bactericidal activity, a plant extract, a metal ion, an oxidizer, an antioxidant, or a combination thereof.

The particle-forming composition may be prepared using a microfluidic system comprising an aqueous stream and an ethanolic stream having flow rate ratio between the aqueous and ethanolic streams of from 2:1 to 5:1, or a flow rate ratio of about 3:1. For example, cholesterol, egg L-α-phosphatidylglycerol (EPG), and L-α-phosphatidylcholine (EPC) may be present in the ethanolic stream. In certain embodiments, the aqueous stream may comprise the releasable agent.

A method of inhibiting a target bacteria is described herein which comprises exposing the target bacteria to the bacteria-responsive particles, which may be prepared according to the described method.

The following abbreviations, among others, may be used herein: CDC, cholesterol-dependent cytolysins; CHOL, Cholesterol; EPG-Na, egg phosphatidylglycerol-sodium; EPC, egg L-α-phosphatidylcholine or “egg phosphatidylcholine”; PPG, propylene glycol; VLY, vaginolysin (a CDC from G. vaginalis); AMP, antimicrobial peptide; PLY, pneumolysin (a CDC from S. pneumoniae); EE, encapsulation efficiency; SPC, soybean phosphatidylcholine; LC, loading capacity; PS, particle size; PDI, polydispersity index; DLS, dynamic light scattering; ZP, zeta potential; FRR, alcohol-to-aqueous flow rate ratio; TFR, total flow rate; MBC, minimum bactericidal efficacy; MIC, minimum inhibitory concentration; PES, polyethersulfone; IPD, invasive pneumococcal disease; PBS, phosphate buffered saline.

A system is described that can release an agent (cargo/payload/drug) when in contact with bacteria that produce cholesterol-dependent cytolysins (CDCs). The particles are cholesterol rich, and their preparation is described herein.

The particles (referenced herein interchangeably as a system/formulation, or composition) may be referred to as a bacteria-responsive “platform”. The release rate of the agent can be altered based on the characteristics and composition of the particles. For food packaging applications, certain preferred components may be included in such particles, as well as combinations of such agents. Combinations of particle components and the use of a plurality of agents within a particle or system are envisioned.

An exemplary embodiment of the particles, as described herein in Example 1 in the embodiment of liposomes, permits detection of Gardenerella vaginalis, and the liposome releases the payload (calcein) in the presence of the cytolysin, vaginolysin (VLY). Calcein is described as an exemplary payload because it is a fluorescent molecule that can be easily monitored.

Another exemplary embodiment described herein in Example 2 permits detection of Streptococcus pneumoniae. The particles are liposomes, and the liposomes release the payload (nisin Z) in the presence of the cytolysin, pneumolysin (PLY). Nisin Z as the payload is an antimicrobial peptide which may be used for therapeutic purposes.

Films and surface coatings are prepared in Example 3.

The Releasable Component or Agent. A wide variety of releasable components are possible, and may be referred to as “components”, “agents”, “active agents”, “drugs”, “payload” or “cargo”. More than one may be used. The releasable component may be an active component or may be inert or passive. The releasable component may include one or more of an antioxidant, a colour indicator such as a compound that changes colour when a bacteria or bacterial product is present, a processing aid having an intended effect upon release, a passive agent that is detectable, a preservative, a bactericide, a broad spectrum bactericide, a fungicide, a virucide, an antimicrobial agent such as an organic acids, a bacteriocins, a protein toxin produced by bacteria or archaea, an enzyme, an essential oil such as one possessing bactericidal capacity, a plant extract, a metal ion, an oxidizer, or any combination of these. The agent may be food-grade, such as those acceptable for use in food packaging industry applications; cosmetic-grade for cosmetic applications; or pharmaceutical-grade, as may suit the intended application.

Applications. The food packaging industry utilizes polymers, plastics, glass, metal, and paper, which may or may not come into contact with the food contained therein. Clamshell packaging, plastic trays, wraps, bags, bottles, jars, cans, tins, boxes, and coated surfaces of paper, are some non-limiting examples of food packaging types amenable to use of the particles/formulation and platform, as described herein. Aside from food packaging surfaces being coated, the formulation may be used for additional applications such as for coating any surface that may benefit from on-demand release of an agent, such as an antimicrobial agent, when certain bacteria are present. Such surfaces include high-contact surfaces such as countertops; curtains, drapes, or blinds; medical equipment or surfaces within infection-prone areas of hospitals, care facilities, or food preparation facilities. A film or application-ready coating containing such particles as described herein are envisioned.

Cholesterol-Dependent Cytolysins (CDCs). A wide variety of cholesterol-dependent cytolysins (CDCs) are known, and may be the component to which the particle responds to release the agent or cargo, as described herein. Examples of CDCs include, but are not limited to the following. Clostridium CDCs include tetanolysin from C. tetani; botulinolysin from C. botulinum; perfringolysin O from C. perfringens; septicolysin O from C. septicum; histolyticolysin O from C. histolyticum; novyilysin from C. Novyi A (oedematiens); chauveolysin from C. chauvoei; bifermentolysin from C. bifermentans; sordellilysin from C. sordelli. An example of an Arcanobacterium CDC is pyolysin from A. pyogenes. An example of a CDC from Brevibacillus is laterosporolysin from B. laterosporus. An example of a CDC from Gardnerella is vaginolysin from G. vaginalis. An example of a CDC from Lactobacillus is inerolysin from L. iners. An example of a CDC from Paenibacillus is aveolysin from P. alvei. Examples of CDCs from Streptococcus include lectinolysin from S. mitis; intermedilysin from S. intermedius; suilysin from S. suis; pneumolysin from S. pneumoniae; streptolysin O from S. canis; streptolysin O from S. Dysgalactiae subsp. Equisimilis; and streptolysin O from S. pyogenes. Examples of CDCs from Bacillus include cereolysin O from B. cereus; thuringolysin O from B. thuringiensis; and Anthrolysin O from B. anthracis. Examples of CDCs from Listeria include seeligerolysin O from L. seeligeri; ivanolysin O from L. ivanovii; and listeriolysin O from L. monocytogenes. These examples are illustrative of the numerous known CDCs. Responsiveness to CDCs released from microbes, whether or not the CDC is fully characterized, may serve to release the agent from the particle and are envisioned herein.

In addition to other particle components noted herein, the particles may contain natural phospholipids (typically sourced from egg or soybean), such as including neutral phospholipids: phosphatidylcholine (PC) or phosphatidylethanolamine (PE); anionic phospholipids such as phosphatidylserine (PS), phosphatidylglycerol (PG), phosphatidic acid (PA), or phosphatidylinositol (PI). Synthetic phospholipids may be included in the particles, such as neutral phospholipids: phosphatidylethanolamine (DPPE), dipalmitoylphosphatidylcholine (DPPC), 1,2-distearoyl-sn-glycero-3-phosphoethanolamine (DSPE), 1,2-distearoyl-sn-glycero-3-phosphocholine (DSPC), 1,2-Dioleoylphosphatidylethanolamine (DOPE), 1,2-Dioleoyl-sn-glycero-3-phosphocholine (DOPC), hydrogenated Soybean Phosphatidylcholine (HSPC), 1-palmitoyl-2-oleoyl-sn-glycero-3-phosphocholine (POPC), 1,2-Dimyristoyl-sn-glycero-3-phosphocholine (DMPC); and anionic phospholipids that may be synthetic, such as: 1,2-Dipalmitoyl-sn-glycero-3-phosphatidic acid sodium salt (DPPA), dipalmitoylphosphatidylglycerol (DPPG), 1,2-Distearoyl-sn-glycero-3-phosphate (DSPA), and 1,2-Distearoyl-sn-glycero-3-phosphoglycerol (DSPG); or which may be cationic, such as: 1,2-dioleoyl-3-trimethylammonium-propane (DOTAP), 1,2-di-O-octadecenyl-3-trimethylammonium propane (DOTMA), DC-cholesterol (hydrochloride) (DC-Chol), and Dimethyldioctadecylammonium (bromide) (DDAB). Surface ligands may be included in the particles, such as: polyethylene oxide (PEG), hyaluronic acid (HA), polyvinyl acetate (PVA), chitosan, or others including but not limited to proteins, antibodies, and aptamers. Cationic Ionizable Lipids may be included in the particles, such as: (6Z,9Z,28Z,31Z)-Heptatriaconta-6,9,28,31-tetraen-19-yl 4-(dimethylamino)butanoate (DLin-MC3-DMA), 1,2-dioleoyl-3-dimethylammonium-propane (DODAP), [(4-hydroxybutyl)azanediyl]di(hexane-6,1-diyl)bis(2-hexyldecanoate) (ALC-0315), 9-Heptadecanyl 8-{(2-hydroxyethyl) [6-oxo-6-(undecyloxy) hexyl]amino}octanoate (SM-102), 1,2-dioleyloxy-3-dimethylaminopropane (DODMA), N-(4-carboxybenzyl)-N,N-dimethyl-2,3-bis(oleoyloxy) propan-1-aminium (DOBAQ), 1-methyl-4,4-bis[(9Z,12Z)-9,12-octadecadien-1-yloxy]-piperidine (YSK05), N,N-dimethyl-2,3-bis[(9Z,12Z)-9,12-octadecadien-1-yloxy]-1-propanamine (DLin-DMA), N,N-dimethyl-2,2-di-(9Z,12Z)-9,12-octadecadien-1-yl-1,3-dioxolane-4-ethanamine (DLin-KC2-DMA), 5-(dimethylamino)-pentanoic acid, (6Z)-1,2-di-(4Z)-4-decen-1-yl-6-dodecen-1-yl ester (CL1), or 1,2-Distearoyl-sn-Glycero-3-Phosphatidylglycerol sodium (5A2-SC8).

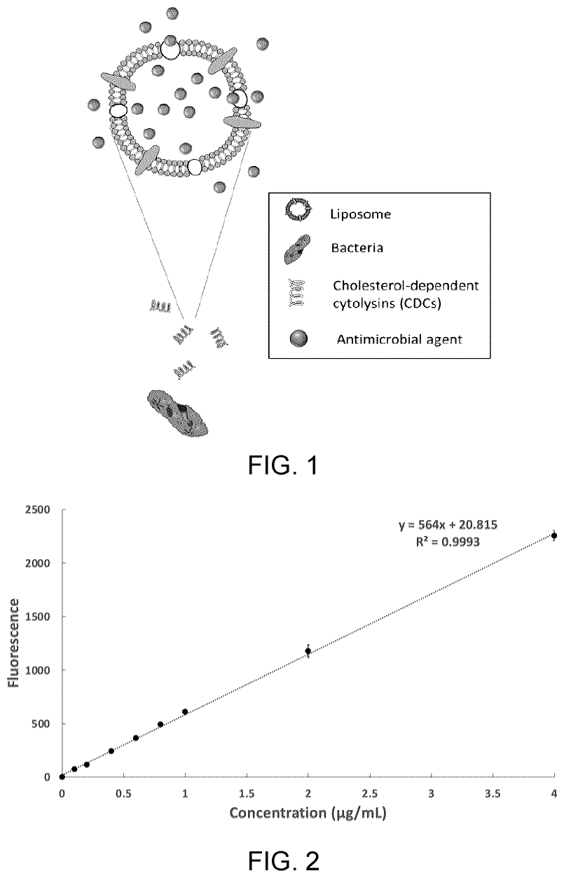

FIG. 1 shows a general schematic of a particle, which is a liposome, formed as described herein. In the presence of a bacteria that releases cholesterol-dependent cytolysins (CDCs), the liposome surface, having surface-exposed cholesterol therein, is rendered permeable to thus release an antimicrobial agent contained within the liposome, so as to counteract or indicate the presence of the bacteria.

### EXAMPLES

### Example 1

### Bacteria-Responsive Drug Release Platform for Food Packaging

### Overview:

World hunger is a huge concern despite the fact that 17% of total global food production is waste. Part of this is due to the short shelf-life of food products resulting in waste with a negative impact on the environment. Designing better and smarter food packaging that can help prolong the shelf-life of food products will dramatically reduce food spoilage and promote food sustainability. Current food products require the inclusion of preservatives to prevent spoilage, improve appearance and maintain the food's nutritional quality. Preservatives only prolong food shelf life and do not eliminate bacteria. By the time bacteria growth is detected, it is too late and the entire food product must be discarded. Furthermore, certain preservatives may have unwanted side effects. As a result, a “smart” bacteria-responsive platform has been developed which only releases antimicrobial agents in the presence of specific bacteria. This reduces the exposure of food products and the consumer to unwanted chemicals while prolonging the shelf-life of food products. Common bacteria involved in food poisoning include bacillus, clostridium, listeria and streptococcus. These bacteria produce a class of virulence factors called cholesterol-dependent cytolysins (CDCs). Four liposomal formulations (average size of 300 nm) were prepared and their responsiveness evaluated to CDCs produced by the model bacteria, Gardnerella vaginalis. The results demonstrate that the liposome formulations achieve sustained drug release over a 5 day period in response to Gardnerella vaginalis and minimal drug release in the presence of Lactobacillus crispatus, a bacteria that does not produce CDCs, indicating the high specificity of the system. Overall, this is the first bacteria-responsive drug release platform utilizing CDCs as the trigger for drug release. Its potential to be used as a surface coating on food packaging, or an integral component of a packaging film, makes it an ideal system for prolonging the shelf-life of consumer products, such as food products, and in reducing unnecessary exposure of preservative or other anti-microbial components to the consumer.

### INTRODUCTION

World hunger is a huge concern despite the fact that 17% of total global food production is wasted [1]. Part of this is due to the short shelf-life of food products resulting in waste with a negative impact on the environment. Designing better and smarter food packaging that can help prolong the shelf-life of food products will dramatically reduce food spoilage and promote food sustainability. The global food packaging market size is currently valued at >$350 billion USD and is expected to expand at a compound annual growth rate of 5.5% from 2022 to 2030 (Food Packaging Market Size & Trends, 2023 [65]).

Current food products require the inclusion of preservatives to prevent spoilage, improve appearance and maintain the food's nutritional quality. Preservatives only prolong food shelf life and do not eliminate bacteria. By the time bacteria growth is detected, it is too late and the entire food product must be discarded. Furthermore, certain preservatives may have unwanted side-effects. For example, studies have shown that benzoic acid may increase hyperactivity in children with attention deficit hyperactivity disorder (Walczak-Nowicka et al., 2022 [66]). Some artificial preservatives such as BHA and BHT may be “reasonably anticipated to be a human carcinogen” as labeled by the National Institutes of Health (RoC Profile, 2021 [67]). Some groups have evaluated the incorporation of antimicrobial agents as a surface coating on food packaging (Fu & Dudley, 2021 [68]). With these technology platforms, antimicrobial agents are released continuously even when it's not necessary. This will expose the food product and consumer to unwanted chemicals. Other groups have evaluated the inclusion of indicators and sensors into the packaging material (Müller et al., 2019 [69]). These types of technologies are good for detecting changes to environmental conditions and quality characteristics of the food product. Unfortunately, they do not eliminate the bacteria responsible for these changes and does not prolong shelf-life.

As a result, a “smart” microbial-responsive platform is developed and described herein, that will only release agents, such as antimicrobial agents (preferably naturally-derived) in the presence of specific bacteria. This will reduce the exposure of food products and the consumer to unwanted chemicals while prolonging the shelf-life of food products. Common bacteria involved in food poisoning include bacillus, clostridium, listeria and streptococcus. These bacteria produce a class of virulence factors called cholesterol-dependent cytolysins (CDCs) [70]. CDCs, as toxins, are capable of binding to cell membranes in the presence of cholesterol (CHOL), forming large and oligomeric complexes, and then inserting into the bilayer to form water-filled pores [70]. Other groups have developed bacteria-responsive systems for treating bacterial infections based on enzymes secreted by the target bacteria such as lipase and hyaluronidase. Unfortunately, these enzymes are commonly included as additives in food products, hence, reducing the specificity of triggered drug release.

In this example, a liposome-based bacteria-responsive drug release platform is described, which is responsive to vaginolysin (VLY), a model CDC [71]. This permits on-demand bacteria-responsive release of an agent. This platform technology has application in food packaging and beyond, such as for surfaces that have potential exposure to bacteria.

### Materials and Methods:

Materials and reagents. Egg L-α-Phosphatidylcholine (EPC), cholesterol (CHOL) and calcein were purchased from Sigma Aldrich. Egg L-α-phosphatidylglycerol (sodium salt) (EPG-Na) was purchased from Sigma Aldrich (Avanti Polar Lipids). Propylene glycol (PPG) was purchased from TCI. Dialysis devices (Spectra-Por® Float-A-Lyzer®G2, molecular weight cutoff 20 kDa) were purchased from Sigma Aldrich. Dialysis tubing (molecular weight cutoff 14 kDa) was purchased from VWR. 0.45 μm polyethersulfone (PES) syringe filter was purchased from Froggabio. Sodium hydroxide was purchased from Sigma Aldrich. Amicon® Ultra-15 centrifugal filters (molecular weight cutoff 3 kDa and 10 kDa) were purchased from Sigma Aldrich. Triton X™-100 was purchased from Sigma Aldrich. Brain heart infusion (BHI) (GranuCult®) and Columbia agar were purchased from Sigma Aldrich. Gardnerella vaginalis (ATCC 14018) Lactobacillus crispatus (ATCC 33820) were purchased American Type Culture Collection (ATCC). Lactobacilli Man-Rogosa-Sharpe (MRS) agar (BD Difco™) and MRS broth were purchased from Thermo Fisher Scientific. Sheep blood (sterile, defibrinated) was purchased from Cedarlane. Yeast extract (Gibco™) and proteose peptone No. 3 (Gibco™) were purchased from Thermo Fisher Scientific. Casman agar base was purchased from BD. N-(2-Hydroxyethyl) piperazine-N′-(2-ethanesulfonic acid), 4-(2-Hydroxyethyl) piperazine-1-ethanesulfonic acid (HEPES) was purchased from Sigma Aldrich. Horse serum (Gibco™, heat inactivated, New Zealand origin) was purchased from Thermo Fisher Scientific. Rabbit blood (sterile, defibrinated) was purchased from Thermo Fisher Scientific. Penicillin/streptomycin solution (Gibco™) was purchased from Thermo Fisher Scientific. 0.22 μm polyethersulfone (PES) syringe filter (sterile) was purchased from Froggabio. Sodium chloride, glucose, agar and glycerol were purchased from Sigma Aldrich.

Liposome synthesis and characterization. Calcein of known concentrations (0-4 μg/mL) was used to generate the calibration curve. The fluorescence intensity was measured using Varioskan™ LUX multimode microplate reader (Thermo Fisher Scientific) (excitation wavelength 490 nm, emission wavelength 520 nm). Linear regression was used to generate a calibration curve. Values were normalized to a blank sample.

Liposomes were synthesized using polyol dilution method²⁶. Four formulations were designed with different composition ratios of egg phosphatidylcholine (EPC), egg phosphatidylglycerol-sodium (EPG-Na) and cholesterol (CHOL) (Table 1). In all formulations, the amount of EPG-Na was fixed at 0.0156 mM (12 mg), and the calculations of other components were based on the molar ratio relative to EPG-Na. Briefly, EPC and EPG-Na were dissolved in propylene glycol (PPG) at 60° C. and cholesterol was dissolved in propylene glycol at 120° C. Once the two solutions were mixed well, calcein stock solution was quickly poured into the mixture and stirred at 60° C. for a few minutes. The suspension was diluted with Milli-Q water (pH adjusted to 7.4) preheated to 60° C. and stirred for 30 minutes. The final concentration of PPG was kept within 3% (w/v). Then, liposomes were sonicated for 5 s on/off for different time periods (80 s, 100 s, 200 s, 400 s) at amplitude 3 using an ultrasonicator (Qsonica™ S-4000) before stirred at 60° C. for 1 hour. The suspension was cooled down to room temperature. Finally, all liposomes were passed through a 0.45 μm PES membrane filter twice.

TABLE 1
Liposome Formulation Design

| | Molar ratio | | | |
|---|---|---|---|---|
| | For- | For- | For- | For- |
| Component | mulation 1 | mulation 2 | mulation 3 | mulation 4 |
| EPC | 10 | 10 | 10 | 10 |
| EPG-Na | 1 | 1 | 1 | 1 |
| CHOL | 9 | 10 | 11 | 12 |
| Mol % of CHOL | 45% | 47.6% | 50% | 52% |

Three methods were applied to separate non-encapsulated calcein from liposomal calcein: centrifugation, ultrafiltration, and dialysis. For centrifugation, liposomes were centrifuged at 40,000 g for 2 hours at 4° C. and washed four times with Milli-Q water (pH 7.4). For ultrafiltration, liposomes were washed seven times using Amicon Ultra centrifugal filter (3 kDa and 10 kDa cutoff) at 4° C. For dialysis, liposomes were dialyzed against 2 L Milli-Q water (pH 7.4) for five days using Slide-A-Lyzer™G2 Dialysis Cassette with membrane molecular weight cutoff (MWCO) of 20 kDa.

Encapsulation efficiency (EE) was calculated both directly and indirectly using the equations below:

$$Encapsulation efficiency ( indirect ) ( \% ) = \frac{\begin{matrix}Amount of initial calcein added - \\ Amount of unencapsulated calcein\end{matrix}}{Amount of initial calcein added} \times 100 \%$$

$$Encapsulation efficiency ( direct ) ( \% ) = \frac{Amount of encapsulated calcein}{Amount of initial calcein added} \times 100 \%$$

For the indirect method, the fluorescence intensity of the supernatants from liposomes encapsulating calcein harvested from centrifugation was measured using Varioskan™ LUX multimode microplate reader (excitation wavelength 490 nm, emission wavelength 520 nm). For the direct method, liposomes were treated with Triton™X-100 to dissolve phospholipids and were then diluted with Milli-Q water (pH 7.4) to achieve a final concentration of Triton™X-100 of 3%. The mixture was incubated at room temperature for 30 minutes prior to the measurements. The influence of Triton™X-100 on fluorescence intensity of calcein was avoided by using 3% Triton™X-100 aqueous solution (pH 7.4) as the blank.

To study the influence of total lipids and initial calcein concentration on EE, parallel formulations were designed. The composition ratio of parallel formulations was based on Formulation 2. EE values are presented as the mean±standard deviation. One-way analysis of variance or T-test was performed with P values of <0.05 considered to be significant.

The average diameter, size distribution and zeta potential of liposomes were measured by dynamic light scattering (DLS) using Zetasizer Nano ZS (ZEN3600, Malvern Instruments). For zeta potential measurement, liposome samples were diluted in Milli-Q water and placed in a DTS1070 cuvette (Malvern Panalytical). Each sample was measured by making six recordings at 25° C.

Bacteria culture. Gardnerella vaginalis (G. vaginalis) strain 14018 was obtained from American Type Culture Collection (ATCC) and cultured in liquid media, i.e., brain heart infusion (BHI) medium and ATCC medium 1685 NYC III, and solid media (agar plate) separately. For each liquid culture, 100 μL of preserved strain was thawed on ice and inoculated into 10 mL broth. The culture was incubated at 37° C. and 5% CO₂ overnight. For each solid culture, a sterile inoculation loop was dipped into a liquid culture and then streaked on the agar plate. The plate was incubated at 37° C. and 5% CO₂ for at least 48 hours to show completely isolated colonies. The optical density (OD) of G. vaginalis was measured using SpectraMax M5 Multimode Plate Reader (Molecular Devices Inc.) at the wavelength of 600 nm.

One colony-forming unit (CFU) was scratched from a fresh agar plate and inoculated into 10 mL broth. 50 μL of the cells was drawn from the broth and diluted to 10-fold, 100-fold, 1,000-fold, 10,000-fold and 100,000-fold separately with 1685 NYC III. Four agar plates were spread with 50 μL of the diluted cells (100× dilution, 1,000× dilution, 10,000× dilution and 100,000× dilution) and 50 μL 1685 NYC III. The plates were incubated at 37° C. and 5% CO₂ for 48 hours. The number of the CFUs counted from the 100,000-fold dilution plate was counted three times and averaged. The concentration of the original broth is calculated using the following equation.

$$Bacteria concentration ( numbers / mL ) = \frac{\begin{matrix}The number of the CFUs counted from the 100 , 000 \times \\ dilution plate \times 1000 μ L \times 100 , 000\end{matrix}}{Inoculation volume ( μ L ) \times 1 mL}$$

Lactobacillus crispatus (L. crispatus) strain 33820 was obtained from ATCC and cultured in liquid (Man-Rogosa-Sharpe, MRS) media and solid media (agar plate) separately. The culture was incubated at 37° C. and 5% CO₂. The counting method for L. crispatus was similar to that for G. vaginalis. Agar plates were inoculated with 100 μL cells and incubated at 37° C. and 5% CO₂ for 24-48 hours.

Bacteria-responsive calcein release of liposomes. Calcein's stability in different media was evaluated by measuring its fluorescence intensity that varied with time in 1685 NYC III medium and G. vaginalis supernatant, respectively. Briefly, calcein was dissolved in the medium to reach a concentration of 10 μg/mL (with 1% v/v penicillin/streptomycin) added. The solution was incubated at 37° C. and shaken at 100 rpm (avoid light). Samples were taken at different time intervals and changes of fluorescence intensity were monitored.

G. vaginalis and L. crispatus were inoculated into 10 mL 1685 NYC III and 10 mL MRS broth, respectively and incubated overnight. The concentrations (CFUs/mL) of G. vaginalis and L. crispatus were kept the same. Bacterial cells were removed through centrifugation at 8,000 g for 10 minutes. 4 mL bacterial supernatant was as mixed with purified liposomes and penicillin/streptomycin (1% v/v) to reach a total volume of 5 mL. 4 mL 1685 NYC III or 4 mL MRS broth mixed with equal amounts of purified liposomes and penicillin/streptomycin (1% v/v) was kept as control group. The reactions were incubated at 37° C. and shaken at 100 rpm (avoid light). 400 μL of reactions were taken at different time intervals (0 hour, 6 hours, 12 hours, etc.) and centrifuged at 40,000 g for 2 hours at 4° C. to remove the pellets. The fluorescence intensity of the supernatant was measured to determine the calcein concentration using calibration curve. To compare the responsive release from G. vaginalis and L. crispatus supernatants, the amount of calcein released from control group (pure medium) was subtracted from experiment group.

### Results and Discussion:

Calcein calibration curve. Calcein was chosen as a model drug to be encapsulated into liposomes as it is relatively easy to be quantified by spectroscopy due to calcein's strong fluorescence. In addition, calcein is a hydrophilic molecule that can be encapsulated inside liposomes instead of inserted into lipid bilayers. This will result in low phospholipid permeation that helps avoid possible drug leakage.

FIG. 2 shows the calcein calibration curve. Known concentrations of calcein (0-4 μg/mL) were used. Fluorescence emission was measured at 520 nm with excitation wavelength at 490 nm. Values represent mean±standard deviation (n=3).

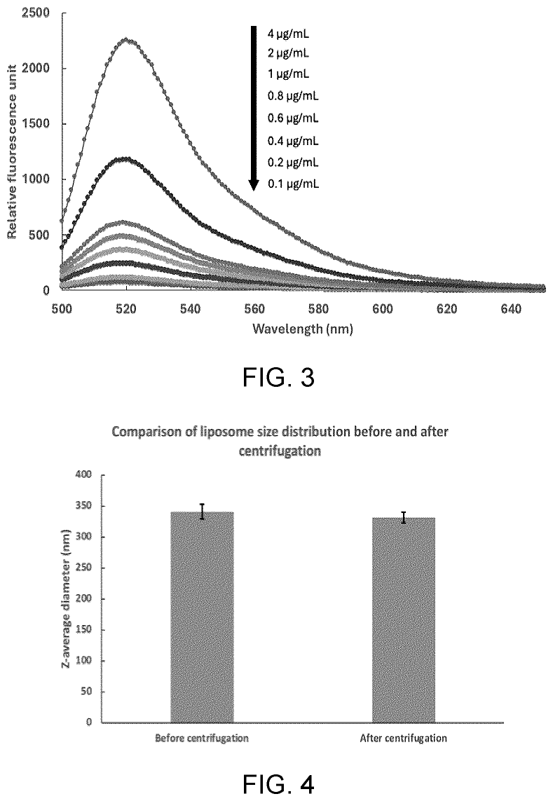

FIG. 3 shows the fluorescence spectra (500-650 nm) of calcein aqueous solutions with different concentration ranging from 0.1 μg/mL to 4 μg/mL. (Excitation wavelength: 490 nm).

Polyol dilution method was chosen to prepare liposomes due to its unique advantages over other conventional strategies such as thin film hydration and reverse-phase evaporation. Propylene glycol is water-soluble and non-toxic compared to volatile organic solvents such as chloroform. Thus, there is no need to remove solvents from the final liposomal suspension because propylene glycol, once applied does not harm human tissues or cells, nor the microbiome of human skin, gut or other tissues, such as microbes normally found in the vagina. Moreover, propylene glycol also helps improve liposome stability during long-term storage due to the interaction between polyols and lipid membrane. Therefore, polyol dilution is a simple and valuable method of preparing liposomes encapsulating drugs.

For components selection in liposome formulations, EPC was chosen as the structural component in terms of cost-effectiveness, stability and superior drug carrying ability over other types of phospholipids. First, phospholipids from natural sources such as plants (e.g. soybean) and animals (e.g. egg yolk) have a lower product cost than synthetic phospholipids. Second, phospholipids isolated from egg yolk has better oxidation stability compared to those isolated from soybeans due to the relatively high saturation level of egg yolk lecithin, resulting in more controlled quality production of liposomes. In addition, it has been proved that liposomes made from EPC demonstrate higher EE and lower drug leakage rate than those made from soybean phosphatidylcholine (SPC) when it comes to drug loading.

CHOL was incorporated into liposomes with different molar ratios ranging from 45% to 52% as the bacteria-responsive component to evaluate liposome drug release performance in the presence of vaginolysin (VLY). Another function of CHOL is to increase bilayer rigidity of liposomes and reduce membrane permeability. EPG-Na was introduced at a relatively low molar ratio as a negatively charged lipid to prevent potential liposome aggregation by creating electrostatic repulsion between particles.

FIG. 4 shows the Z-average diameter values are the means of six repeat measurements (±standard deviation) (P value >0.05) of liposomes prepared by sonication for 200 s at amplitude 3 using a sonicator. The liposomal suspension was centrifuged at 40,000 g for 2 hours at 4° C. to remove non-encapsulated calcein. The liposomes harvested from centrifugation was completely resuspended in Milli-Q water (pH 7.4) prior to DLS measurement.

Three purification methods were compared in terms of effectiveness and efficiency to determine an optimal condition to separate non-encapsulated calcein from liposomes. It is found that centrifugation at a moderate centrifugal force (40,000 g) is the best option without sacrificing liposome stability compared to ultrafiltration and dialysis. After four repeated sequent washing, the calcein concentration of the supernatant from liposomes is less than 0.1%. The average diameters and size distributions of liposomes before and after purification through centrifugation were measured by DLS (FIG. 4). There is no significant difference in size distribution, indicating that liposomes remain relatively stable without much aggregation.

Ultrafiltration was applied as the second purification method, but it turned out that it was not very effective in achieving complete separation. Liposomal suspension was filtrated with a 10 kDa cutoff membrane filter under centrifugation conditions of 5000 g at 4° C. followed by seven repeat sequent washing. However, the calcein concentration of the filtrate was still more than 5% even after multiple washing steps. More important, increasing washing times results in unnecessary loss of liposomes, as liposomes could be forced to squeeze through the membrane and migrate into the filtrate. This assumption was further affirmed by DLS that the filtrate had an average size of less than 200 nm that indicates the presence of some small liposomes passed through. To prevent liposomes migration, a membrane filter with 3 kDa cutoff was used for filtration. However, the filtration efficiency was greatly compromised, with centrifugation time nearly tripled. Increasing centrifugal force was not applicable, either, as the membrane could be damaged. Similarly, dialysis was also not the ideal purification method as it took as long as a week to remove the non-encapsulated calcein as completely as possible, with a frequent change of the dialysis media to ensure molecule diffusion caused by concentration gradient.

Table 2 provides a comparison of encapsulation efficiency (EE) of six liposome formulations.

TABLE 2
Comparison of Encapsulation Efficiency (EE) of liposome formulations.

| | Total lipids | Amount of | EE (indirect, | EE (direct, |
|---|---|---|---|---|
| Formulation | (mM) | initial calcein | %) | %) |
| F2_1 | 0.11 | 2 mg/mL, 300 μL | 5.73 ± 3.3 | 5.24 ± 1.5 |
| F2_2 | 0.22 | 2 mg/mL, 300 μL | 9.31 ± 2.6 | 6.50 ± 2.8 |
| F2_3 | 0.33 | 2 mg/mL, 300 μL | 9.90 ± 3.0 | 8.67 ± 2.2 |
| F2_4 | 0.33 | 4 mg/mL, 300 μL | 16.03 ± 2.0 | 13.88 ± 2.2 |
| F2_5 | 0.33 | 2 mg/mL, 2 mL | 9.80 ± 2.6 | 8.93 ± 1.5 |
| F2_6 | 0.33 | 4 mg/mL, 2 mL | 15.33 ± 2.1 | 12.83 ± 1.8 |

EE values represent the means of three preparations ± standard deviation.

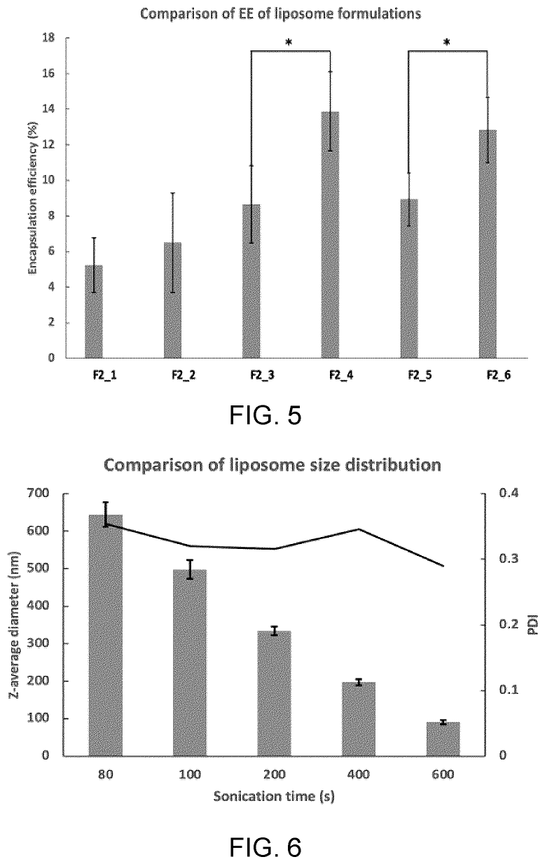

FIG. 5 illustrates encapsulation efficiency (%) EE of liposome formulations (F2_1-F2_6) calculated using direct method. F2_1: 0.11 mM lipids, 300 L 2 mg/ml initial calcein; F2_2: 0.22 mM lipids, 300 μL 2 mg/mL initial calcein; F2_3: 0.33 mM lipids, 300 L 2 mg/ml initial calcein; F2_4: 0.33 mM lipids, 300 μL 4 mg/mL initial calcein; F2_5: 0.33 mM lipids, 2 mL 2 mg/mL initial calcein; F2_6: 0.33 mM lipids, 2 mL 4 mg/mL initial calcein. All values represent the means of three preparations±standard deviation. (P value <0.05).

It is found that EE depends on the total amount of lipids and initial calcein concentration. Comparisons are made among EE calculated using direct method, as indirect method is more susceptible considering possible calcein loss during purification process. Generally, EE increases as the amount of total lipids increases (FIG. 5). In addition, a higher EE can be achieved if the initial calcein solution is more concentrated, as there is significant difference between 2 mg/mL and 4 mg/mL. Although increasing the amount of initial calcein (e.g. from 300 μL to 2 mL) does not result in a significant difference in terms of EE, it helps increase the amount of calcein encapsulated. In this study, the formulation parameters of F2_6 was chosen for Formulation 2, as it favors a larger amount of encapsulated calcein instead of a higher EE, the former of which helps better evaluate the performance of bacteria-responsive calcein release. Therefore, 2 mL 4 mg/mL calcein stock solution was added to Formulations 1-4 as the initial dose. In Formulation 2, the total amount of lipids was set at 0.33 mM, whereas in Formulation 1, 3 and 4 the composition ratio of EPC to EPG-Na was kept the same as that in Formulation 2 with varying amounts of CHOL.

Table 3 shows encapsulation efficiency (EE) of liposome formulation 1-4, as evaluated by different methods.

TABLE 3
Encapsulation Efficiency (EE) of Liposome Formulation 1-4

| | For- | For- | For- | For- |
|---|---|---|---|---|
| EE (%) | mulation 1 | mulation 2 | mulation 3 | mulation 4 |
| Indirect method | 14.5 ± 2.7 | 15.33 ± 2.1 | 12.67 ± 3.2 | 10.66 ± 2.5 |
| Direct method | 13.40 ± 2.1 | 12.83 ± 1.8 | 11.92 ± 2.3 | 10.79 ± 1.1 |

All values represent the means of three preparations ± standard deviation

Size distribution and zeta potential of liposomes. The size distributions of liposomes treated with varying sonication time lengths were measured by DLS to study the effect of sonication on size.

FIG. 6 shows the size distribution of liposomes with varying sonication time lengths ranging from 80 s to 600 s without using PES filter. The Z-average diameter values are the means of six repeat measurements (±standard deviation).

All liposome preparations were not filtered with the PES membrane filter. In general, the average diameter of liposomes decreases as sonication time length increases. However, liposomes with relatively large diameter (>500 nm) were less stable according to the quality report of Zetasizer measurement results, with larger size distribution standard deviation and varying zeta potential values. In contrast, smaller particles tend to be more stable in that the measuring results of size distribution and zeta potential are relatively consistent. But purification efficiency is greatly sacrificed. For example, particles as small as 100 nm were very hard to be spun down even with a centrifugal force of 100,000 g. Hence, sonication time was fixed at 200 s to prepare liposomes with an average diameter ranging from 300 nm to 400 nm, so that liposomes can be purified through centrifugation at 40,000 g without compromising drug loading amount and stability too much. Once sonication time was determined, Formulations 1~4 were filtered with 0.45 μm PES membrane filter to further narrow the size distribution prior to the measurement.

TABLE 4
lists size distribution and zeta potential of formulations 1-4.
Size Distribution and Zeta Potential of Formulations 1-4

| Formulation | Size distribution | Zeta potential |
|---|---|---|
| 1 | 319.3 ± 4.8 nm, PDI 0.252 | −64.0 ± 0.9 mV |
| 2 | 324.1 ± 3.9 nm, PDI 0.255 | −56.3 ± 1.6 mV |
| 3 | 330.1 ± 2.7 nm, PDI 0.286 | −59.2 ± 0.7 mV |
| 4 | 318.9 ± 3.5 nm, PDI 0.284 | −64.5 ± 0.7 mV |

Gardnerella vaginalis culture optimization. Optimizations were performed to both liquid culture and solid culture. It is found that G. vaginalis is unable to grow in BHI probably due to the lack of nutrients such as serum, whereas it grows well in 1685 NYC III. In addition, the growth rate of G. vaginalis also depends on the total amount of inoculation. The more cells are inoculated, the faster G. vaginalis grows. Finally, 100 μL of preserved cells was determined as the optimal inoculation amount to allow for a suitable growth rate in broth culture.

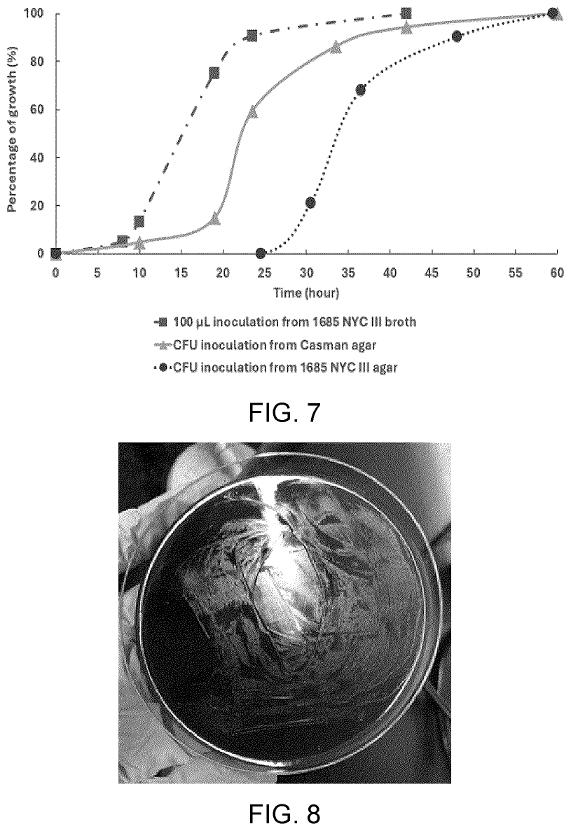

FIG. 7 shows G. vaginalis growth (OD₆₀₀) in liquid culture. 100 μL cells were inoculated into 10 mL broth and incubated at 37° C. Alternatively, one CFU was scratched from the plate and inoculated into 10 mL broth and incubated at 37° C. Values represent the mean of three recordings.

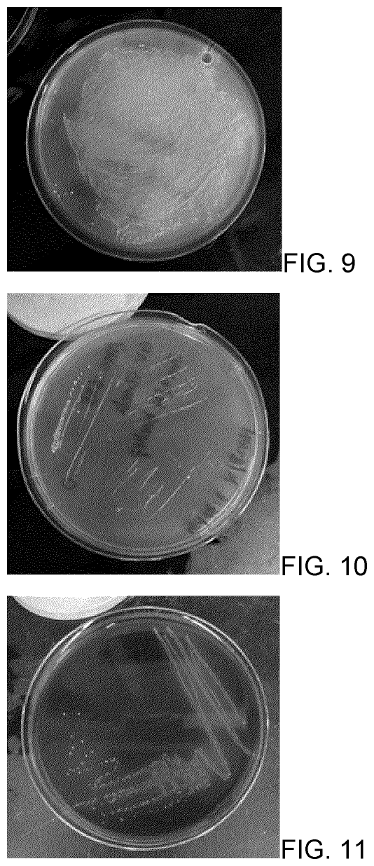

The growth of G. vaginalis on agar plates was found to be very poor at the beginning. Therefore, a series of optimization studies were carried out to determine an optimal cultivation condition for G. vaginalis, including agar plate selection, inoculation method and inoculation amount (Table 5). No bacteria growth was observed on Columbia agar plates even after a week's incubation (FIG. 8), whereas growth was observed in varying degrees on both 1685 NYC III and Casman agar plates. The results showed that spreading method could facilitate a heavy and confluent growth, leading to bacteria lawn formation (FIG. 9), while streaking method were prone to result in separately grown colonies (FIG. 10 and FIG. 11). Apart from inoculation method, bacteria growth also depends on inoculation amount. Too much inoculation is very likely to bring about fierce competition among bacteria colonies, leading to very few or no growth. Overall, Casman agar was considered more suitable than Columbia agar and 1685 NYC III agar. Thus, for bacteria counting, G. vaginalis was cultured on Casman agar with streaking method and incubated at 37° C. for 48 hours.

TABLE 5
Solid culture optimization of G. vaginalis

| | | Inoculation | |
|---|---|---|---|
| | Inoculation | volume | |
| Agar plate type | method | (μL) | Bacteria growth |
| Columbia agar | Spreading | 100 | FIG. 8 shows Columbia agar |
| (5% sheep blood) | | | plate. No bacteria growth was |
| | | | observed after one week′s |
| | | | cultivation. |
| Columbia agar | Spreading | 50 | No growth. |
| (5% sheep blood) | | | |
| Columbia agar | Streaking | N/A | No growth |
| (5% sheep blood) | | | |
| 1685 NYC III | Spreading | 30 μL | FIG. 9 shows 1685 NYC III |
| agar | | | agar plate (spreading). |
| | | | Bacteria lawn formed |
| | | | after 48 hours′ cultivation. |
| 1685 NYC III | Streaking | N/A | FIG. 10 shows 1685 NYC III |
| agar | | | agar plate (streaking). |
| | | | Very few CFUs were found |
| | | | after 48 hours′ cultivation. |
| Casman agar | Streaking | N/A | FIG. 11 shows Casman agar |
| (5% rabbit blood) | | | plate (streaking). CFUs were |
| | | | found after 36-48 hours′ |
| | | | cultivation. |
| Casman agar | Spreading | 100 μL | No growth. |
| (5% rabbit blood) | | | |

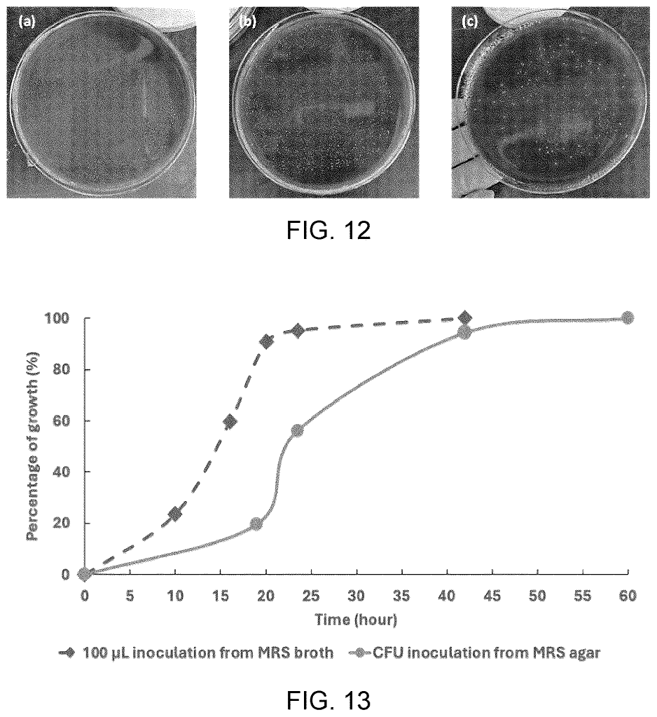

FIG. 12 shows CFUs of G. vaginalis grown on Casman agar plates (5% rabbit blood). Panel a) 1,000-fold dilution; Panel b) 10,000-fold dilution; and Panel c) 100,000-fold dilution. For each plate, 50 μL bacterial cells and 50 μL 1685 NYC III medium were spread.

FIG. 13 shows L. crispatus growth (OD₆₀₀) in liquid culture. 100 μL cells were inoculated into 10 mL broth and incubated at 37° C. Alternatively, one CFU was scratched from the plate and inoculated into 10 mL broth and incubated at 37° C. Values represent the mean of three recordings.

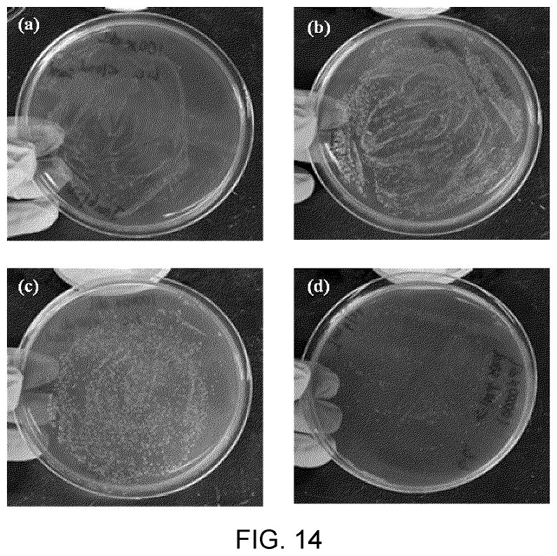

FIG. 14 shows CFUs of L. crispatus grown on MRS agar plates. Panel a) 100-fold dilution; Panel b) 1,000-fold dilution; Panel c) 10,000-fold dilution; and Panel d) 100,000-fold dilution. For each plate, 100 μL bacterial cells were spread.

Both (i) G. vaginalis and (ii) L. crispatus were counted on the 100,000-fold dilution plates of FIG. 12 and FIG. 14. For (i) G. vaginalis: the number of the 100,000-fold dilution plate was counted to be 249. Hence, the original concentration of the broth was 4.97×10⁸/mL, which corresponded to 78.6% of the total bacteria growth (20 hours' cultivation). ii) For L. crispatus: the number of the 100,000-fold dilution plate was counted to be 325. Hence, the original concentration of the broth was 6.5×10⁸/mL, which corresponded to 56.3% of the total bacteria growth (16 hours' cultivation).

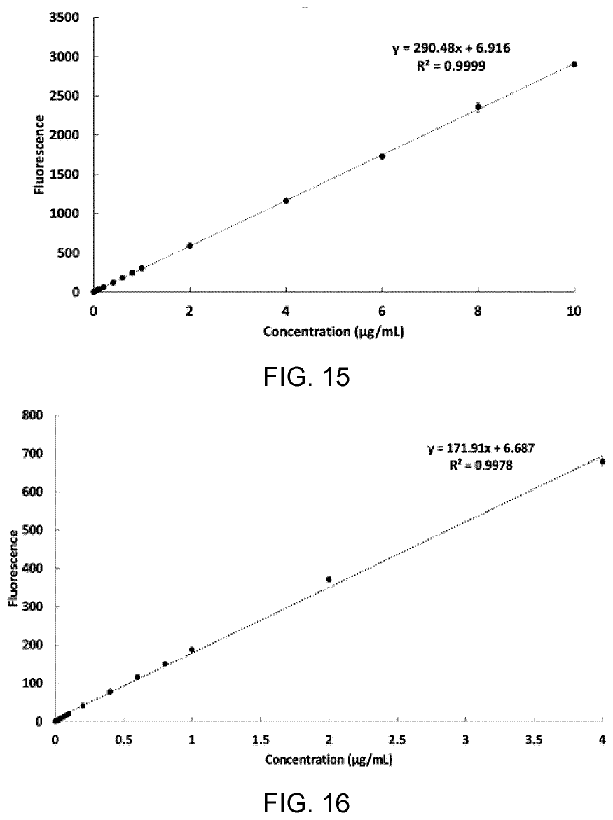

Bacteria-responsive calcein release of liposomes. FIG. 15 shows a calcein calibration curve in 1685 NYC III medium. Known concentrations of calcein (0-10 μg/mL) were used. Fluorescence emission was measured at 520 nm with excitation wavelength at 490 nm. Values represent mean±standard deviation (n=3).

FIG. 16 shows a calcein calibration curve in MRS medium. Known concentrations of calcein (0-4 μg/mL) were used. Fluorescence emission was measured at 520 nm with excitation wavelength at 490 nm. Values represent mean±standard deviation (n=3).

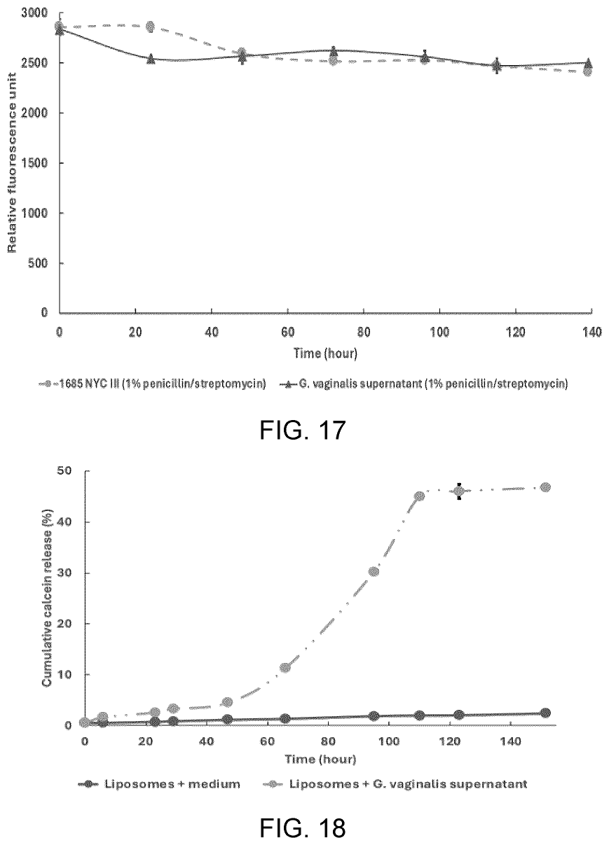

FIG. 17 shows calcein fluorescence stability in 1685 NYC III medium and G. vaginalis supernatant. The initial concentration of calcein is 10 μg/mL.

Calcein was dissolved in 1685 NYC III medium and G. vaginalis supernatant separately. The fluorescence intensity was monitored for 140 hours (FIG. 17). To prevent potential contamination during the course, penicillin and streptomycin were added to the medium to inhibit microorganism growth.

After 140 hours, 1685 NYC III medium and G. vaginalis supernatant with 1% penicillin/streptomycin were both clear without turbidity, indicating that 1% penicillin/streptomycin was effective enough to prevent bacteria growth. Therefore, 1% penicillin/streptomycin addition was adopted in the following studies. Moreover, 11-15% of calcein fluorescence decrease was found during the process. The negligible quench makes calcein a suitable indicator of responsive release from liposomes.

The calcein release profiles of Formulations 1~4 were studied to evaluate liposomal responsiveness to G. vaginalis. The bacteria were grown to reach an OD value of around 1.5 before the supernatant was collected and mixed with liposomes, During this period, the bacteria are in the late progress of exponential phase in which the production of vaginolysin is supposed to be maximized. As is shown in FIG. 18 to FIG. 21, four formulations were able to achieve a 3-5 days' sustained release in the presence of G. vaginalis supernatant, whereas four control groups demonstrated negligible release. The responsive release is attributed to the damage of liposome structure caused by vaginolysin produced by G. vaginalis.

FIG. 18 pertains to Formulation 1, showing cumulative calcein release stimulated by G. vaginalis. The values represent mean±standard deviation (n=3).

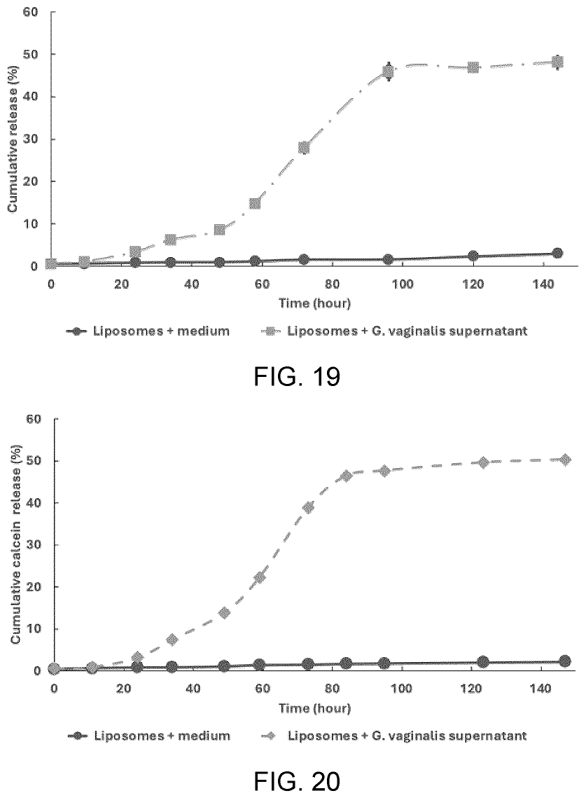

FIG. 19 relates to Formulation 2, showing cumulative calcein release stimulated by G. vaginalis. The values represent mean±standard deviation (n=3).

FIG. 20 pertains to Formulation 3, showing cumulative calcein release stimulated by G. vaginalis. The values represent mean±standard deviation (n=3).

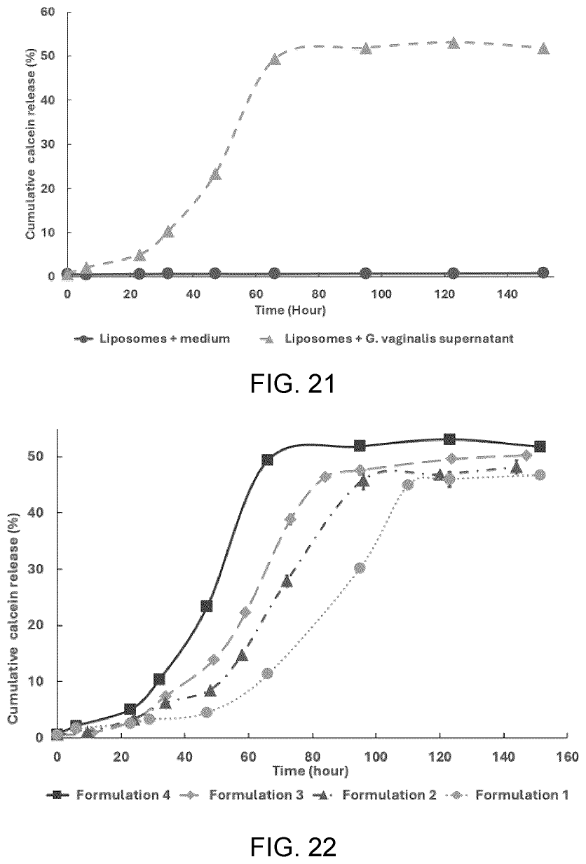

FIG. 21 relates to Formulation 4, showing cumulative calcein release stimulated by G. vaginalis. The values represent mean±standard deviation (n=3).

FIG. 22 shows a comparison of cumulative calcein release stimulated by G. vaginalis. The values represent mean±standard deviation (n=3).

The calcein release rate depends on the amount of CHOL in liposomes, as more CHOL promotes vaginolysin binding to the bilayer and thus pore formation, resulting in calcein discharge. The accumulative calcein release percentage is slightly different among four formulations, ranging from 46.7% to 51.8% (FIG. 22). It is plausible that Formulation 4 demonstrates the largest accumulative release percentage as more liposomes are disrupted by vaginolysin with CHOL concentration as high as 52 mol %.

The calcein release profile in the presence of L. crispatus supernatant was also evaluated to verify liposomes' inertness to L. crispatus. L. crispatus were cultivated to reach an OD value of around 1.5 to make sure that the L. crispatus broth had a similar concentration with G. vaginalis broth based on numbers of colonies per milliliter prior to responsive calcein release studies.

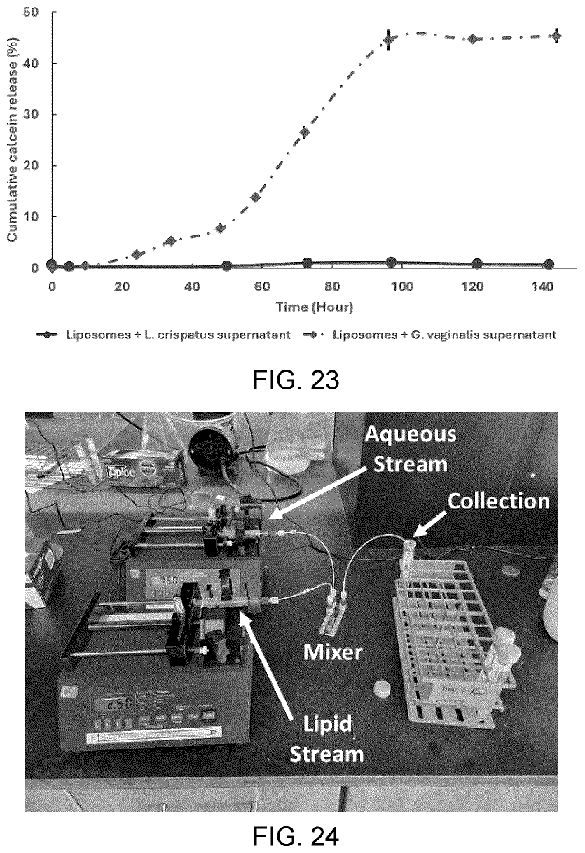

FIG. 23 shows cumulative calcein released from Formulation 2 in the presence of G. vaginalis supernatant or L. crispatus supernatant. The amount of calcein released from pure medium was subtracted to eliminate the difference caused by medium. The values represent mean±standard deviation (n=3). As is shown in FIG. 23, liposomes showed minimal responsive release in the presence of L. crispatus supernatant. This means that liposomes would not harm normal microbiota, such as is found in a woman's vagina.

### Conclusion

In this study, a bacteria-responsive drug release platform triggered by the presence of CDCs has been successfully developed. Four formulations of particles, in this case liposomes, encapsulating calcein were prepared via the polyol dilution method, which is facile and non-cytotoxic. A series of optimizations in formulation design and purification methods were performed to determine an optimal protocol. The optimized EE was calculated to be 10-15%. The prepared liposomes had an average diameter of around 300 nm with a relatively narrow size distribution (the PDI value ranging from 0.25 to 0.28). The average zeta potential of the liposomes is around −60 mV. All the results indicate that the preparation method is reliable, and that the liposomes can remain stable due to electrostatic repulsion. All four liposomal formulations demonstrated drug release only in the presence of G. vaginalis (produces CDCs) and not in the presence L. crispatus (does not produce CDCs). In the presence of G. vaginalis supernatant, the liposomes were capable of achieving sustained calcein over a 5 day period, while in the presence of L. crispatus, the liposomes showed negligible calcein release. However, the average cumulative calcein release slightly varied among the four formulations, ranging from 46.7% to 51.8%, which might be attributed to the difference in the molar ratio of CHOL. The amount of calcein released increased as the amount of CHOL increases, suggesting that VLY produced by G. vaginalis can insert into the liposome membrane and create pores in the presence of CHOL, resulting in calcein release.

The CDCs naturally secreted by bacteria act as the trigger to release the agent, such as a release of an active agent or drug, in this case calcein. This example further illustrates that this platform has numerous applications involving on-demand agent or drug release, especially with applications in food packaging, and for the coating of other surfaces. Consumer products with a limited shelf-life can benefit from a wide variety of applications.

### Example 2

### Microfluidic Development of a Pneumolysin-Responsive Liposomal Platform for Selective Treatment of Streptococcus Pneumoniae

### Overview:

Targeting of bacteria-produced virulence factors has been gaining interest due to the potential for more responsive and localized delivery and treatment. As such, the present study aimed to develop cholesterol-rich liposomal formulations capable of responsive release of therapeutic and neutralization of the cholesterol-dependent cytolysin (CDC) pneumolysin (PLY), produced by the infectious pathogen S. pneumoniae. To successfully produce these formulations, a microfluidic system was designed and a Box-Behnken response surface methodology was employed to optimize several key microfluidic parameters with regard to encapsulation efficiency (EE), loading capacity (LC), particle size (PS), and polydispersity index (PDI). A model was then utilized and validated to optimize the independent variables given set targets, leading to a flow rate ratio between the aqueous and ethanolic stream of 3.01:1, a 61.07 μg/mL concentration of nisin Z antimicrobial peptide therapeutic, and a 3.85% molar composition of the lipid egg phosphatidylglycerol (EPG) with an overall composite desirability of 0.99. The sequestration capabilities of these liposomes ranging from 30-75 mol % cholesterol content were then examined via hemolytic assays with both purified toxin and S. pneumoniae bacterial culture, with full protection optimized at a cholesterol-rich level of ≥60 mol %, compositions due to the presence of sufficient free surface cholesterol. Drug release assays also demonstrated that PLY toxin was capable of liposomal destabilization and subsequent burst, releasing encapsulated nisin at elevated cholesterol levels, but efficacy was observed to be reduced when CHOL is below 60 mol %, with levels of ≥45 mol % having efficacy under the tested conditions. These results suggest these liposomes, and this liposomal system can serve as a potent and selective release platform, such as a therapeutic platform for S. pneumoniae treatment by prevention of growth thereof, with applications extending to other pathogenic CDC-producing bacteria.

### INTRODUCTION

The Gram-positive S. pneumoniae is an opportunistic pathogen that maintains a commensal relationship with the host when colonized in mucosal surfaces of the human upper respiratory tract, with over 97 characterized serotypes [2A]. This asymptomatic presence in the upper respiratory tract allows for rapid transmission within a community via aerosols and can eventually lead to infection within the individual [2B]. Invasive pneumococcal disease (IPD) involves spreading of the bacterium to normally sterile bodily fluids, with examples being sepsis, bacteraemic pneumonia, septic arthritis, and meningitis [3]. The most prevalent pneumococcal disease is pneumococcal pneumonia, caused by migration of the bacterium to the lungs, with S. pneumoniae believed to be responsible for 55% of all lower respiratory tract infection deaths in 2015 [4,5].

The effectiveness of S. pneumoniae arises due to the range of produced colonization and virulence factors as described in Table 6, which serve to give insight towards disease pathogenesis and, consequently, potential points of treatment and intervention. This has been an area of growing interest, as it has been found that these virulence factors are typically essential for growth and highly specific to the host bacterium. These traits make them promising therapeutic targets, as it both limits selection pressure towards resistant strains while also reducing damage to the commensal microbiota of the host through more specific targeting [6]. The virulence factors of S. pneumoniae have implications ranging from adherence, colonization, bacterial spreading, and avoidance of host immune defenses, serving to highlight the importance of understanding their mechanisms when designing a therapeutic platform [7].

TABLE 6
Pneumococcal virulence factors and their role in pathogenesis

| Virulence Factor | Location | Primary Function | Reference |
|---|---|---|---|
| Capsule | Polysaccharide | Aids in adherence | [8] |
| | layer on | and inhibits | |
| | cell wall | phagocytosis | |
| Biofilm | Surrounding | Protects bacteria | [9] |
| | bacterium | and reduces | |
| | | recognition by host | |
| Pili | Cell surface | Inhibits phagocytosis and | [10] |
| | | promotes colonization | |
| Choline- | Surface of | Aids binding to host cell | [11] |
| Binding | cell wall | receptors and | |
| Proteins | | protects against | |
| | | the complement system | |
| Metal-Binding | Surface of | Transports magnesium | [12] |
| Proteins | cell wall | and zinc into cytoplasm | |
| Other Surface | Surface of | Inhibits complement | [7] |
| Proteins | cell wall | and act as adhesins | |
| Neuraminidase | Surface of | Degrades mucus to | [13] |
| | cell wall | improve adherence | |
| Lytic Amidase | Intracellular | Breaks down bacterial | [14] |
| | | peptidoglycan to | |
| | | release toxins | |
| Bacteriocin | Secreted by | Inhibit growth of | [15] |
| | bacteria | competing bacteria | |
| Pneumolysin | Cytoplasm | Pore formation and | [16] |
| (PLY) | | cell lysis | |

Of the aforementioned virulence factors, pathogenesis of S. pneumoniae is largely controlled by release of the protein pneumolysin (PLY), which is part of a pore-forming toxin family referred to as cholesterol-dependent cytolysins (CDCs). Over 20 species of pathogenic bacteria have been associated with the production of CDC toxins that demonstrate largely conserved structure and function. These protein toxins act mainly to recognize and bind to cholesterol-rich regions within cellular membranes, at which point they reassemble into a pore complex and insert into the lipid bilayer to cause membrane disruption [17]. PLY can act in concert with proteases, invasions, and adhesins to damage membranes, modify inflammation, aid bacterial escape, and increase host-to-host transmission of pneumococcal disease [16]. Bacterial genomics have indicated that the most promising vaccine and treatment targets against S. pneumoniae include PLY, PspA, and LytA, as mutations in the corresponding genes have led to reduced pneumococcal virulence [18]. By virtue of its pore-forming properties, PLY is lytic to cells at high concentrations but at sublytic concentrations, such as during early infection, it can also induce proinflammatory reactions and activate the host complement [19]. The amount of PLY released into the cytoplasm has been found to vary between 20-700 ng/ml depending on infection severity, but is most commonly reported around or below 200 ng/ml [20,21]. This heterogeneity in expression depends on the location and stage of pathogenesis of the bacterium, with the highest PLY expression often found during colonization of the nasopharynx and virulent spread to induce IPD due to its metabolically demanding nature [22].

To combat S. pneumoniae infection, conventional antibiotic therapies include macrolides, fluoroquinolones, or β-lactams either on their own or in combination, but their effectiveness is limited due to 15-30% of all strains being classified as multi-drug resistant [23]. Further, antibiotic therapy faces long development cycles, low bioavailability, barrier challenges in pulmonary delivery, and adverse side effects [24]. To combat this, there has been the development of three major vaccines, including a 23-valent pneumococcal polysaccharide vaccine (PPSV23) and both a 13- and 7-valent pneumococcal conjugate vaccine (PCV) [25]. While highly effective in children under the age of 5, they are still limited by lowered efficacy in aging populations and the increased prevalence of S. pneumoniae strains not actively targeted [26,27]. This has sparked research into alternative therapeutics, including antimicrobial peptides (AMPs), bacteriophages, adjuvants, probiotics, and essential oils [28]. One of the most promising alternatives are AMPs, as they have existed naturally for millennia with limited resistance development and break down into non-toxic metabolites. They typically function by disrupting the bacterial membrane or interacting with intracellular components, and due to their short amino acid sequences they can be easily synthesized [29]. One such AMP, nisin (for example, nisin Z), is an FDA approved 3.5 kDa peptide-derived antibiotic, referred to as a type A “lantibiotic”, that has been widely used as a food preservative over several decades due to its properties as a bacteriocin. However, its applications have recently been extending into the biomedical field due to its anti-biofilm properties and ability to inhibit growth of drug-resistant pathogens [30]. It has displayed strong antimicrobial activity against S. pneumoniae strains through both pore formation and inhibition of peptidoglycan biosynthesis via lipid II binding in the bacterial membrane, but again is limited by poor targeting to infection sites [31].

To address limitations in vaccination and delivery of therapeutic to multi-drug resistant pneumococcal strains, research has been focusing on the utilization of nanomaterials. The ability of these systems to release drugs within the site of infection dramatically increases bioavailability of the therapeutic and reduces off-target effects. Pulmonary inhalation for delivery of nano-systems is desirable as it has potential to increase local targeting and provide faster onset times as compared to systemic delivery [24]. However, this route poses several key barriers, as it has been found that only particle sizes below 260 nm can effectively penetrate deep into alveoli and reduce macrophage clearance [24,32]. Beyond this, hydrophilic and neutral formulations with a strong safety profile are preferred to prevent capture by respiratory mucus. One of the most common nanoparticle systems used for targeted bacterial delivery are liposomes, which are spherical vesicles comprised of one or more concentric phospholipid bilayers (lamellae) and an inner aqueous core. This imparts a structural similarity to bacterial membranes, giving liposomes a unique ability to initiate fusion and promote high bactericidal potency while still being non-toxic in nature [33].

Due to the natural incorporation of cholesterol in their structure, liposomes were a natural choice for developing a PLY-responsive nanosystem. However, conventional preparation techniques require self-assembly of the lipids in the bulk phase, often resulting in heterogeneous and multilamellar particles that require post-processing techniques [34,35]. As such, microfluidic development of liposomes has been gaining recent attention. This technique produces liposomal particles through mixing of an ethanolic phase with dissolved lipid solution mixing with an aqueous phase containing therapeutic. The microstructures within the chip induce the formation of helical and chaotic flow in the laminar stream, significantly accelerating mixing between the two phases [36]. The diffusion of the alcohol stream in water will eventually reach the lipids solubility limit, causing them to precipitate and form intermediate micelles that subsequently become liposomes as their bilayer structure is energetically favourable. The rate of mixing and precipitation kinetics can be modulated through variation of the alcohol-to-aqueous flow rate ratio (FRR) and the total flow rate (TFR) of the system [37]. Other relevant parameters for a given formulation are the liposomal constituents, which commonly include phospholipids and sterols, post-processing conditions, and concentrations of both the lipid and therapeutic used, with a general summary present in Table 7. Modulation of these properties can control the lamellarity of the synthesized liposomes, with unilamellar structures often being preferred for drug delivery applications due to their smaller diameter and improved encapsulation efficiency [38]. Overall, microfluidics have demonstrated stronger control over particle size distribution and structure as compared to conventional techniques, and have seen recent clinical success in industrial settings [39].

TABLE 7
Relevant parameters during development of a microfluidic workflow

| Component | Purpose | Reference |
|---|---|---|
| Mixer | Induces mixing between two streams | [36] |
| | to accelerate liposome formation | |
| Tubing | Generates a water-tight flow | [37] |
| | Length should be minimized to reduce | |
| | void volume and compatible with solvents | |
| Lipid | Significant implications on liposome | [38] |
| Components | structure and properties | |
| | Various phospholipids, sterols, | |
| | and other additives | |
| Lipid | Influences concentration of | [40] |
| Concentration | liposomes in solution | |
| | Has implications on particle lamellarity | |
| Therapeutic | Controls the amount of therapeutic in solution | [41] |
| Concentration | Impacts encapsulation efficiency | |
| | when in excess to liposomes | |
| TFR | Total speed both fluid streams are pumped at | [37] |
| | Faster mixing speeds can reduce liposome size | |
| FRR | Volumetric ratio of aqueous to | [42] |
| | organic fluid stream | |
| | Impacts liposome concentration and | |
| | shortens diffusion length in mixer | |
| Buffer | Added to dilute residual ethanol content | [43] |
| | Can influence liposomal stability | |
| | and mediate pH-responsive release | |
| Buffer | Concentration of liposome solution | [44] |
| Exchange | with reduced ethanol content | |
| | Depending on method utilized, can | |
| | have impact on particle properties | |
| Filtering | Removes contaminants and induces sterility | [45] |
| | Can improve homogeneity | |
| | of liposomes but can also | |
| | cause loss of sample due to filter interactions | |
| Storage | Impacts hydrolysis rate and overall | [46] |
| | long-term stability of liposome solutions | |
| | Storage type (eg. powder, | |
| | aqueous) impacts particle | |
| | quality and therapeutic encapsulation | |

A major benefit for nanoparticle targeting of PLY is that the toxin shows little variation between different pneumococcal strains and serotypes [47]. However, while PLY has been of interest as a vaccine candidate, it is not expressed on the pneumococcal capsule but rather found in the supernatant after release, making antibody-induced opsonization more difficult [48]. As such, there has been recent interest in developing nano-based systems capable of efficiently reaching pneumococcal infection sites to facilitate PLY interaction. These studies have utilized nanomaterials to detect [49,50] and sequester the toxin [51-56], but to date there are no studies on PLY-induced therapeutic release for the treatment of S. pneumoniae, despite similar release kinetics proven to be effective for other CDCs [57,58]. As such, the present study aims to develop a microfluidic system for formulation of cholesterol-rich liposomes capable of PLY-responsive release in proximity to S. pneumoniae, acting both to sequester and inactivate the toxin as well as produce bactericidal effects.

### Experimental:

### Microfluidic Preparation of Liposomes

Experimental Microfluidic Setup. The developed microfluidic system, as displayed with the Experimental setup shown in the photograph of FIG. 24, utilized two dual channel syringe pumps (NE-4000, New Era Pump Systems Inc., Farmingdale, NY USA) connected to a microfluidic mixer chip (labelled “Mixer”) (MIX-4, PreciGenome LLC, San Jose, CA USA), which featured an internal herringbone geometry. Three pieces of PTFE tubing with a 1/16″ outer diameter and 1/32″ internal diameter (Fluorostore, Parsippany, NJ USA) were cut to exactly 21 mm to minimize the amount of dead volume and attached to the inlets and outlet of the chip. All ports were fitted with a series of Idex Leur adapters and PFA flangeless fittings at a 1/16″ outer diameter (Cole-Parmer, Vernon Hills, IL USA) to ensure a watertight system. Two 15 mL syringes were then loaded with the aqueous phase (labelled “Aqueous Stream”) and ethanolic phase (labelled “Lipid Stream”), respectively. The aqueous phase contained nisin Z (TOKU-E, Bellingham, WA USA) dissolved in neutral Milli-Q water at a known concentration, while the ethanolic phase contained the lipids egg L-α-phosphatidylglycerol (EPG) (Avanti Polar Lipids Inc, Alabaster, AL USA), cholesterol, and L-α-phosphatidylcholine harvested from egg yolk (EPC) (Millipore Sigma, Burlington, MA USA) to a final lipid concentration of 20 mM. Each lipid solution was warmed in a water bath set to 50° C. to ensure full dissolution in ethanol prior to mixing at the desired molar concentrations. The syringes were then tilted and flicked vertically to remove any air bubbles that may hinder the mixing process and the liquid was pushed manually to the edge of the outlet. These syringes were then securely mounted on the syringe pumps and connected to the tubing, at which point the speed and volume to pump were set accordingly to match a total flow rate of 6 mL/min and flow rate ratio varying between 2:1 and 4:1 for aqueous:ethanol content for a total 1.5 mL formulation volume. Injection of the solutions were then initiated simultaneously and the liposomal solution was collected from the outlet in a reservoir (labelled “Collection”). Newly formed liposomes underwent a series of wash cycles with Milli-Q water through the use of Amicon Ultra-15 centrifugal filter units (MWCO 30 kDa, Millipore Sigma, Burlington, MA USA) spun at 3000×g in 10 min cycles. This was continued until a 15× dilution of the original formulation volume was reached in order to lower residual ethanol content, at which point it was concentrated back down to 1 mL. This solution was then collected and syringe filtered through 0.45 μm PES membranes (Montreal Biotech Inc, Dorval, QC CA) to remove contaminants and sterilize the liposomes, at which point they were stored at 4° C. until use.

Box-Behnken Design. A three-level, three-factor Box-Behnken design was utilized to optimize liposomal formulations for desired outputs of the dependent variables using Minitab® 16.0 software. This response surface design consists of a series of points located at each midpoint and center of a multidimensional cube to produce a series of equations capable of giving theoretical outputs. These second-order polynomial models follow the form of Eq. (1):

$$Y = {A}_{0} + {\sum }_{i = 1}^{k} {A}_{i} {X}_{i} + {\sum }_{i = 1}^{k} {A}_{ii} {X}_{i}^{2} + {\sum }_{i = 1}^{k - 1} {\sum }_{j = 2}^{k} {A}_{ij} {X}_{i} {X}_{j} \qquad ( 1 )$$

where X represents the independent factors, Aₒ, Aᵢ, Aᵢᵢ, and Aᵢⱼ are regression coefficients of constant, linear, quadratic, and interactions terms respectively, and k are the total number of independent factors. The tested independent variables (X) were the flow rate ratio of the aqueous and ethanol stream during mixing, nisin concentration, and EPG molar composition as expressed in Table 8. These were evaluated by the following dependent variables (Y): encapsulation efficiency (EE), loading capacity (LC), particle size (PS), and polydispersity index (PDI), with experiments performed in 15 randomized runs and repeated in triplicate. Coefficients were then determined via least square regression followed by analysis of variance (ANOVA) to generate a series of polynomial equations capable of describing the relation between each independent variable and the responses. A series of contour plots were then developed with continuous hold variables set to their median value.

This model was then employed to optimize the system according to desired response values, with the final formulation being produced in triplicate and compared to predicted outputs. For all testing, the total cholesterol content was fixed at 60 mol %, while varying EPG content was accounted for through modulation of EPC content.

Where X represents the independent factors, Aₒ, Aᵢ, Aᵢᵢ, and Aᵢⱼ are regression coefficients of constant, linear, quadratic, and interactions terms respectively, and k are the total number of independent factors. The tested independent variables (X) were the flow rate ratio of the aqueous and ethanol stream during mixing, nisin concentration, and EPG molar composition as expressed in Table 8. These were evaluated by the following dependent variables (Y): encapsulation efficiency (EE), loading capacity (LC), particle size (PS), and polydispersity index (PDI), with experiments performed in 15 randomized runs and repeated in triplicate. Coefficients were then determined via least square regression followed by analysis of variance (ANOVA) to generate a series of polynomial equations capable of describing the relation between each independent variable and the responses. A series of contour plots were then developed with continuous hold variables set to their median value.

This model was then employed to optimize the system according to desired response values, with the final formulation being produced in triplicate and compared to predicted outputs. For all testing, the total cholesterol content was fixed at 60 mol %, while varying EPG content was accounted for through modulation of EPC content.

TABLE 8
Variables and levels in Box-Behnken design

| | | Code levels | | |
|---|---|---|---|---|
| Independent Variables | Symbol | −1 | 0 | 1 |
| Flow rate ratio (aqueous:ethanol) | X₁ | 2:1 | 3:1 | 4:1 |
| Nisin concentration (μg/mL) | X₂ | 30 | 60 | 90 |
| PG molar composition (mol %) | X₃ | 1 | 4 | 7 |

### Liposome Characterization

Measurement of Particle Characteristics. Dynamic light scattering (DLS)

(Zetasizer Ultra Red, Malvern Panalytical, Worcestershire, UK) was utilized to determine PS via intensity based mean particle diameter measurements and the molecular weight distribution of the suspension through PDI. Particle electrophoresis conducted in the same instrument reported the zeta potential (ZP) of the suspension. For all measurements, the liposomes were diluted 1:80 in milli-Q water.

Encapsulation Efficiency. Encapsulation efficiency was determined utilizing a 3-(4-carboxybenzoyl) quinoline-2-carboxaldehyde (CBQCA) assay (Molecular Probes, Eugene, OR USA) with a procedure from the manufacturer with some modifications. Briefly, 8.25 μL of liposomal suspension was mixed with 16.5 L of 2% Triton™ X-100 (Sigma Aldrich, St. Louis, MO USA), 115.5 μL of 0.05 M sodium borate (MP Biomedicals, Irvine, CA USA), 156.75 UL of Milli-Q water, 11 μL of 20 mM potassium cyanide (Millipore Sigma, Burlington, MA USA), and 22 μL of 2 mM ATTO-TAG CBQCA reagent (ABP Bioscience, Rockville, MD USA). The solution was vortexed to mix and incubated for 1 hr at room temperature away from light, with fluorescence measurements conducted at an emission and excitation wavelength of 550 nm and 465 nm, respectively. Liposomal dilution in the assay was accounted for based on the initial volume of the suspension and converted to an experimental mass of collected nisin based on a calibration curve made with free drug in suspension. This was compared with the initial mass of drug loaded in the aqueous phase to calculate a direct encapsulation efficiency as outlined in Eq (2). Following this, LC was determined using the aforementioned mass of nisin collected in the formulation as compared to the weight of liposomes recovered following microfluidic processing as displayed in Eq (3):

$$EE ( direct ) = ( \frac{Mass of nisin in formulation}{Initial mass of nisin} ) \times 100 \qquad ( 2 )$$

$$LC = ( \frac{Mass of nisin in formulation}{Mass of recovered liposomes} ) \times 100 \qquad ( 3 )$$

Bacterial Strains and Growth Conditions. Stock cultures of the antibiotic-resistant 23F strain of S. pneumoniae (ATCC 700669, Manassas, VA USA) were kept in a 15% glycerol solution supplemented with brain heart infusion (BHI) broth (Millipore Sigma, Burlington, MA USA) and stored at −80° C. Prior to hemolytic or antibacterial testing, the bacterial strain was streaked onto a BHI agar plate supplemented with 5% defibrinated sheep blood (Thermo Fisher Scientific, Waltham, MA USA) and grown statically for 24 hr at 37° C. and 5% CO₂. Several individual colonies were then transferred to sterile BHI broth and grown for 24 h in the same conditions until an optical density of ~0.4 was achieved as per measurements at 600 nm. This correlates to the end of log phase growth, and at this point the suspension was diluted to achieve a 0.5 McFarland standard (corresponding to ~1.5×10⁸ CFU/mL).

Hemolytic Activity Assay. For hemolytic assays, washed human red blood cells (RBCs) (Innovative Research, Novi, MI USA) were suspended in 1×PBS (pH 7.2, Wisent, Quebec City, QC Canada) to a final concentration of 2%. For sequestration studies, liposomal samples were diluted in PBS to the desired concentration and added to the erythrocytes. The hemolytic reaction was then initiated through the addition of either bacterial supernatant normalized to a 0.5 McFarland standard or recombinant S. pneumoniae serotype 2 PLY (E. coli host expression, MyBioSource, San Diego, CA USA) at the desired volume or concentration. For assays involving PLY, the toxin was preincubated with 2 mM dithiothreitol (DTT) for 1 hr at room temperature prior to the assay in order to reduce the cysteine residue present in the undecapeptide sequence and activate the protein [59]. The solution was incubated in a 1.5 ml Eppendorf microtube for 30 min at 37° C. and non-lysed RBCs were pelleted via centrifugation at 1000×g for 10 min at 4° C. The supernatant was then collected and transferred to a 96 well plate, with the extent of hemoglobin release determined via absorbance measurements at 450 nm. A control set was also included, involving a negative control of RBCs in only PBS and a positive control of RBCs in a 0.02% Triton™ X-100 solution (100% lysis). Percent lysis was determined from these readouts as per Eq (4):

$$Hemolysis ( \% ) = \frac{{OD}_{450} ( sample ) - {OD}_{450} ( negative control )}{{OD}_{450} ( positive control ) - {OD}_{450} ( negative control )} \qquad ( 4 )$$

Liposomal Release Assay. To measure the rate of release of nisin Z from PLY toxin interaction, liposomes were added to a microcentrifuge tube at a concentration of 200 μM. PLY toxin was preincubated with 2 mM DTT for 1 hr to activate the protein structure and then added at a final concentration of 4 μg/mL, with the total volume of the assay brought up to 200 μL through addition of PBS. The tubes were then immediately incubated at 37° C. for varying timepoints, at which point they were collected and transferred to Amicon Ultra-0.5 centrifugal filters (MWCO 30 kDa, Millipore Sigma, Burlington, MA USA). These were immediately spun at 10,000×g for 5 min at 4° C. to separate out released nisin Z. The concentrated solution remaining in the top filter was collected with PBS and 165 L of this solution was then transferred for use in CBQCA protein measurements as previously described without the inclusion of Milli-Q water. A negative and positive control were included for each liposomal series, with the negative control containing just liposomes and PBS and the positive containing a 0.2% Triton™X-100 solution to fully disrupt the particles. The fluorescence readouts were converted to a mass through use of a calibration curve, with percent release calculated through comparison to the previously determined total nisin mass in each sample.

Minimum Inhibitory Concentration. To determine the minimum inhibitory concentration (MIC) the broth microdilution method was utilized. Bacteria grown overnight and standardized to a 0.5 McFarland standard were added at a final concentration of 5×10⁵ CFU/mL to a 1.5 mL microcentrifuge tube. The assay also included varying concentrations of nisin Z suspended in water and normalized to a final water volume of 30 μL, as well as 318 μL of BHI broth to bring the assay to a final volume of 350 μL. Both a negative and growth control were included, with the former containing no bacterial suspension and the latter containing no therapeutic. The tubes were then incubated at 37° C. in a 5% CO₂ environment for 24 hr with subsequent transfer to a 96-well plate and absorbance readings at 600 nm. MIC values were taken as the lowest therapeutic concentration that produced an absorbance below or equal to the negative growth control after incubation.

Statistics. All experiments were performed in triplicate in three independent experiments.

### Results and Discussion

Liposomal Optimization. A microfluidic system capable of producing liposomes was designed as previously described. In brief, the system comprises two dual channel syringe pumps attached to a microfluidic chip with an internal herringbone geometry to induce chaotic flow within the mixer. In a given microfluidic system, there are various parameters that have a significant impact on particle properties. As such, it was necessary to conduct a Box-Behnken optimization design with independent variables of flow rate ratio, nisin concentration, and EPG molar content. For all formulations, the amount of cholesterol was kept constant at 60 mol %, with the complete design and results visible in Table 9.

TABLE 9
Complete Box-Behnken design

| Run | X₁ | X₂ | X₃ | Y₁ - EE (%) | Y₂ - LC (%) | Y₃ - Size (nm) | Y₄ - PDI |
|---|---|---|---|---|---|---|---|
| 1 | −1 | −1 | 0 | 71.94 ± 8.85 | 2.01 ± 0.25 | 146.18 ± 16.09 | 0.14 ± 0.05 |
| 2 | 1 | −1 | 0 | 82.87 ± 7.40 | 5.59 ± 0.50 | 136.50 ± 8.50 | 0.09 ± 0.06 |
| 3 | −1 | 1 | 0 | 70.94 ± 9.14 | 5.96 ± 0.77 | 182.46 ± 5.75 | 0.18 ± 0.02 |
| 4 | 1 | 1 | 0 | 32.30 ± 8.18 | 6.54 ± 1.66 | 303.58 ± 18.14 | 0.24 ± 0.11 |
| 5 | −1 | 0 | −1 | 19.32 ± 9.37 | 1.08 ± 0.53 | 152.71 ± 13.50 | 0.13 ± 0.02 |
| 6 | 1 | 0 | −1 | 4.42 ± 2.71 | 0.60 ± 0.37 | 163.41 ± 23.24 | 0.18 ± 0.06 |
| 7 | −1 | 0 | 1 | 69.46 ± 9.30 | 3.88 ± 0.52 | 143.66 ± 10.88 | 0.18 ± 0.04 |
| 8 | 1 | 0 | 1 | 79.19 ± 5.39 | 10.64 ± 0.73 | 151.91 ± 10.18 | 0.25 ± 0.02 |
| 9 | 0 | −1 | −1 | 24.66 ± 7.49 | 1.13 ± 0.34 | 141.33 ± 10.96 | 0.14 ± 0.02 |
| 10 | 0 | 1 | −1 | 9.27 ± 5.05 | 1.15 ± 0.63 | 167.51 ± 21.46 | 0.19 ± 0.05 |
| 11 | 0 | −1 | 1 | 71.80 ± 6.21 | 3.27 ± 0.28 | 115.70 ± 1.72 | 0.17 ± 0.04 |
| 12 | 0 | 1 | 1 | 75.20 ± 8.95 | 10.27 ± 1.22 | 175.11 ± 11.40 | 0.34 ± 0.01 |
| 13 | 0 | 0 | 0 | 78.57 ± 6.96 | 7.17 ± 0.64 | 140.76 ± 7.14 | 0.17 ± 0.03 |
| 14 | 0 | 0 | 0 | 72.44 ± 7.60 | 6.61 ± 0.69 | 154.53 ± 9.99 | 0.17 ± 0.02 |
| 15 | 0 | 0 | 0 | 68.99 ± 8.41 | 6.30 ± 0.77 | 146.32 ± 3.51 | 0.17 ± 0.03 |

The developed polynomial equations (not shown) showed good fit to the acquired data, as the correlation coefficients (r²) for EE, LC, PS, and PDI were 0.9711, 0.9878, 0.8859, and 0.9570 respectively.

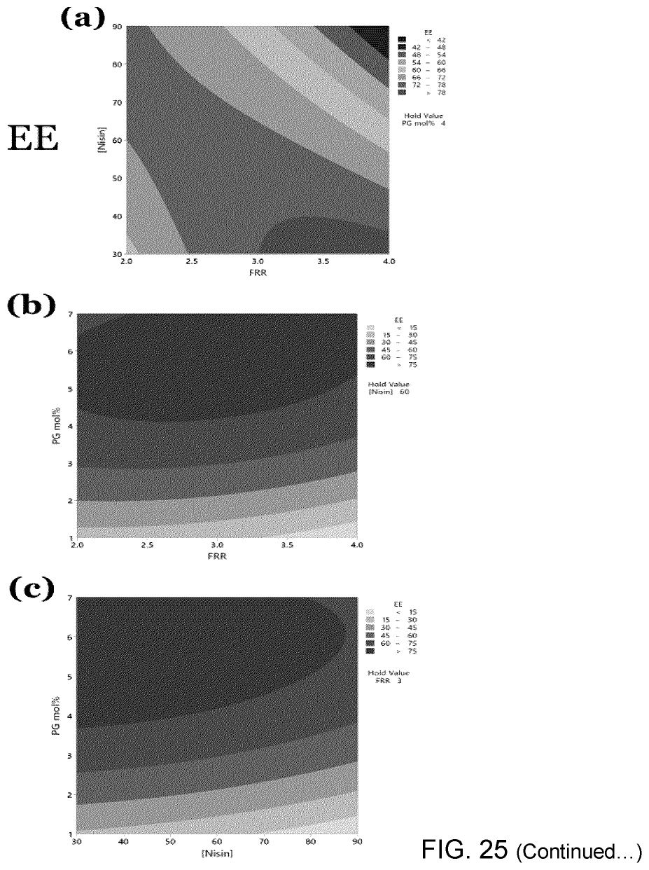

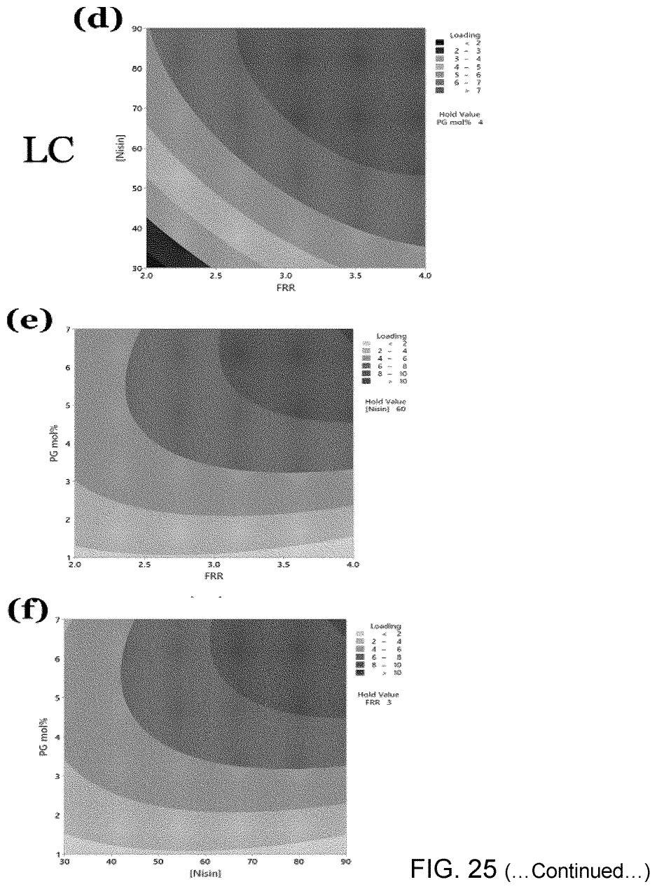

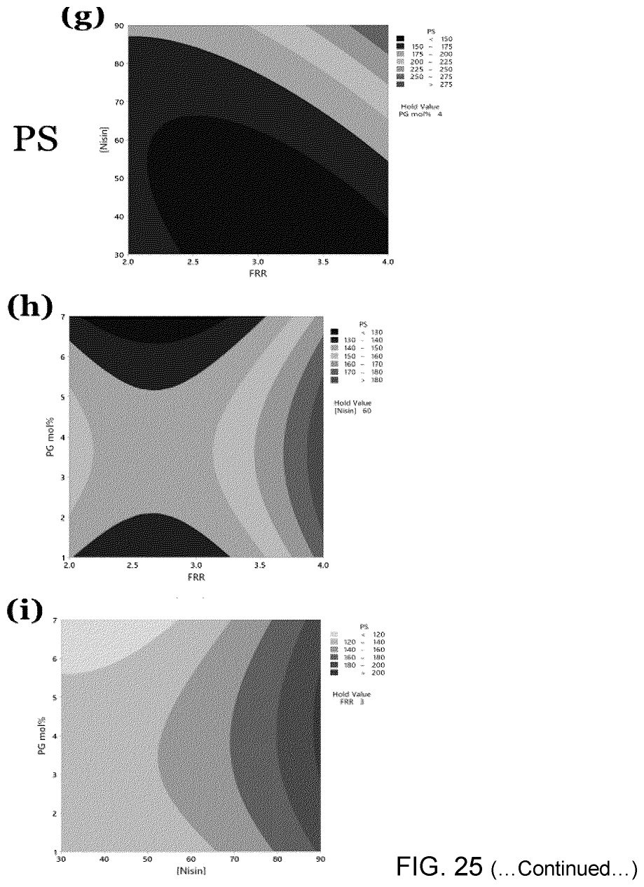

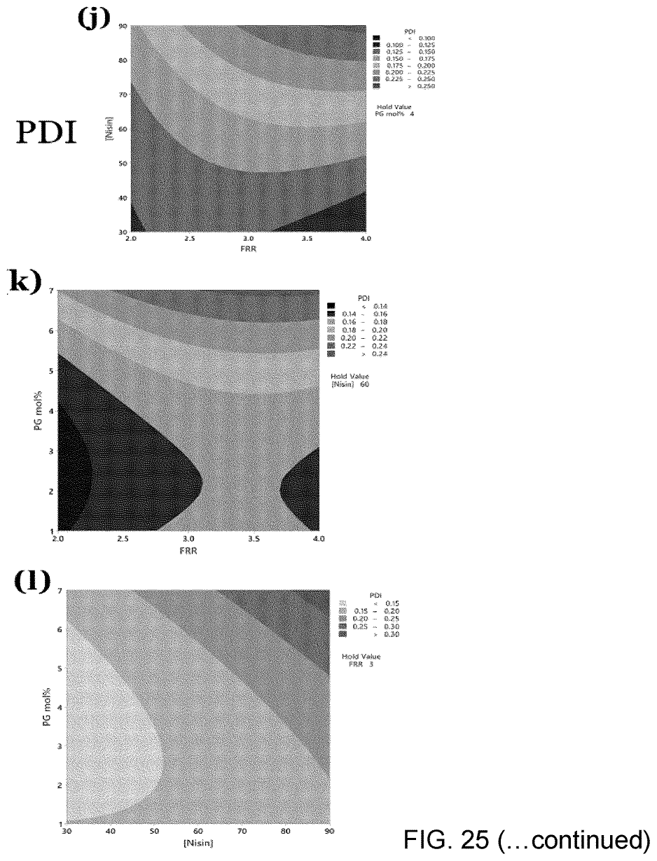

FIG. 25 provides contour plots in Panels a to l, highlighting the influence and interplay between different independent variables. Namely, these examine the correlation between nisin concentration and FRR (Panels a, d, g, j), PG molar concentration and FRR (Panels b, e, h, k) and PG molar concentration and nisin concentration (Panels c, f, i, l). Specifically, the contour plots give indications on the impact these variables had on EE (Panels a-c), LC (Panels d-f), PS (g-i), and PDI (Panels j-l). All hold values were set to their median coded level.

To visualize the relationship between the dependent and independent variables, a series of contour plots were generated in FIG. 25. The EE data (FIG. 25, Panels a-c) suggests that increasing the concentration of nisin Z served to lower encapsulation, indicating the possibility of liposomal saturation within the internal aqueous core. Unsurprisingly, increasing the EPG content led to improved EE due to the charge interactions between the cationic nisin molecules and the anionic phospholipid while FRR had less impact in general. For LC (FIG. 25, Panels d-f), there appeared to be a linear relationship between all three factors, with higher values corresponding to greater drug loading. The interactions between the variables became more complex with regards to PS (FIG. 25, Panels g-i), although it can be noted that increasing nisin concentration increases size of the particle and overall dispersity, likely owing to greater drug loading causing liposomal swelling and aggregation. PG incorporation had a lessened impact on particle size but in general increased PDI (FIG. 25, Panels j-l), while lower FRRs appeared to be optimal to reduce PS and showed little correlation with changes in PDI.

The developed model was then used to predict an optimum response given set target values for the studied variables. These target values, correlating to EE, LC, PS, and PDI values of 72%, 6.60%, 145.0 nm, and 0.17 respectively, had weight and importance values of 1 and computed through Minitab® software. The optimized output for this model indicated a FRR, nisin concentration, and PG molarity of 3.01:1, 61.07 μg/mL, and 3.85 mol % respectively. This led to a strong composite desirability of 0.99, and following this the optimized formulation was fabricated and compared to the models expected outputs as seen in Table 10. Overall, relatively strong agreeance was observed for all response variables and indicates success of the model for accurately predicting liposomal properties given a set of formulation parameters.

TABLE 10
Response results of the optimized formulation and agreeance
as compared to the predicted model outputs

| Response | Observed | Model Prediction | Agreeance (%) |
|---|---|---|---|
| EE (%) | 84.04 ± 1.54 | 71.42 ± 4.64 | 85.0 |
| LC (%) | 7.84 ± 0.14 | 6.60 ± 0.35 | 84.2 |
| PS (nm) | 147.9 ± 8.62 | 148.9 ± 13.9 | 99.3 |
| PDI | 0.19 ± 0.01 | 0.1701 ± 0.01 | 89.5 |

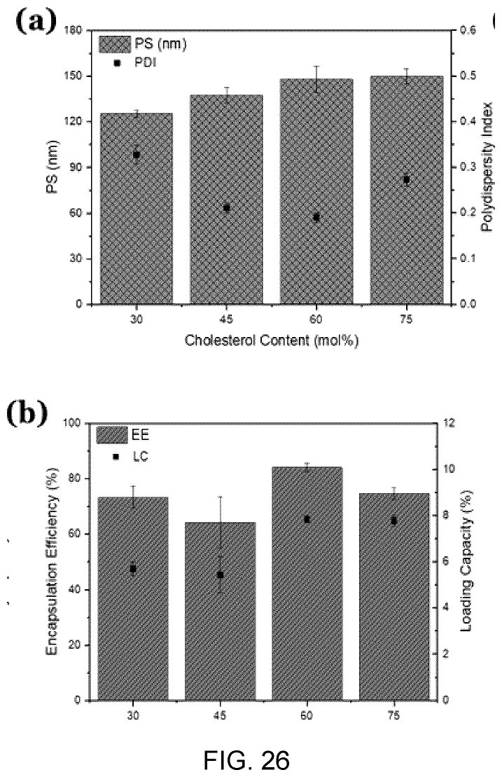

Using the previously elucidated optimal formulation parameters, the mol % of cholesterol was then varied and compared in terms of the previously mentioned response variables as seen in FIG. 26.

FIG. 26 shows the impact of cholesterol content on liposomal properties, namely Panel (a) the PS and PDI, as well as Panel (b) the EE and LC. Cholesterol led to a minor increase in particle size at higher mol % inclusion, as well as the lowest observed PDI at 60 mol % in FIG. 26, Panel (a). This is likely owing to the ability of hydrophobic cholesterol to interact with acyl chains present in the phospholipids, with their inflexible nature imparting rigidity to the membrane. This acts to stabilize the saturated fatty acids and modulate fluidity to promote spherical and monodisperse vesicles [38]. This stabilization effect may also explain why an improved EE was observed at increased cholesterol levels in FIG. 26, Panel (b), as the particles were both more uniform and had an expanded inner aqueous core for drug loading owing to the steric hinderance imparted by cholesterol. Overall, it appears that 60 mol % led to optimal properties of all conditions tested.

Table 11 lists particle composition and characteristics of liposomal formulations fabricated at varying molar compositions of cholesterol (n=3), and is consistent with findings illustrated in FIG. 26.

TABLE 11
Particle composition and characteristics of liposomal formulations
fabricated at varying molar compositions of cholesterol (n = 3)

| | Composition | | | Particle | | | Drug | |
|---|---|---|---|---|---|---|---|---|
| | (mol %) | | | Characteristics | | | Interactions | |
| Form- | | | EPG- | PS | | Zeta | EE | LC |
| ulation | EPC | CHOL | Na | (nm) | PDI | (mV) | (%) | (%) |
| 30 mol % | 66.15 | 30 | 3.85 | 125.27 ± | 0.33 ± | −25.32 ± | 73.33 ± | 5.68 ± |
| | | | | 2.35 | 0.02 | 2.46 | 3.96 | 0.31 |
| 45 mol % | 51.15 | 45 | 3.85 | 137.36 ± | 0.21 ± | −35.73 ± | 64.23 ± | 5.44 ± |
| | | | | 5.04 | 0.01 | 2.41 | 9.35 | 0.79 |
| 60 mol % | 36.15 | 60 | 3.85 | 145.13 ± | 0.17 ± | −33.00 ± | 84.04 ± | 7.84 ± |
| | | | | 7.68 | 0.02 | 7.28 | 1.54 | 0.14 |
| 75 mol % | 21.15 | 75 | 3.85 | 149.84 ± | 0.27 ± | −31.89 ± | 74.69 ± | 7.76 ± |
| | | | | 5.06 | 0.01 | 1.81 | 2.14 | 0.22 |

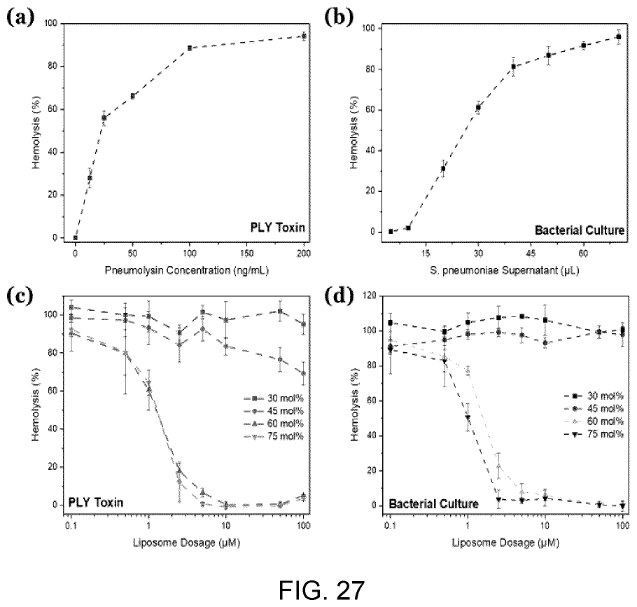

Pneumolysin Sequestration. Purified PLY toxin and the cultured 23F S. pneumoniae strain were compared in their hemolytic capabilities along with an investigation into the sequestration capabilities of the liposomal formulation in FIG. 27.

FIG. 27 shows hemolytic activity of purified PLY toxin and bacterial culture Panels (a)-(b) against human erythrocytes. A concentration capable of inducing full lysis was then used to examine sequestration capabilities of liposomes with varying cholesterol content against purified PLY and bacterial culture Panels (c)-(d).

To begin, recombinant PLY toxin displayed rapid hemolysis in FIG. 27, Panel (a), with full lysis observed at 200 ng/ml. This falls within the range of toxin expression typically found in clinical S. pneumoniae infections [20]. In comparison, hemolytic activity from the bacterial culture had a more delayed response but reached full hemolysis at roughly 70 μL of supernatant, or the equivalent of ~3.0×10⁷ CFU/mL of S. pneumoniae. These results seem to indicate that the bacterial culture is producing PLY toxin but, as can be expected, in a less potent form than purified PLY itself. These values, being 200 ng/ml for the toxin and 70 μL for the bacterial culture, were then chosen to represent full hemolytic activity in subsequent sequestration experiments. As can be seen in FIG. 27, Panel (c, there was little protective effect from liposomes containing less than 60 mol % cholesterol, while the cholesterol-rich formulations exhibited extremely effective toxin sequestration and neutralization at dosages as low as 10 μM. There was little difference separating 60 and 75 mol % compositions, seemingly indicating that a threshold cholesterol content was required to properly facilitate toxin binding. This may stem from studies showing that CDC binding required membranes with surface-exposed cholesterol, which involves a complex interplay between phospholipids and cholesterol [60]. It has been suggested that membrane binding is only triggered when cholesterol concentration exceeds the association capacity of the related phospholipids, as this excess is free to associate with the toxin [61]. A very similar trend was observed for the bacterial culture in FIG. 27, Panel (d), as full erythrocyte protection was also achieved at a liposomal dosage of 10 μM. This supports the previous hypothesis that hemolysis from the bacterial culture was tied to PLY release and not a conjunction of the other various virulence factors produced by the bacteria, as they would not associate with the liposomes in such a fashion. Overall, theses findings suggest strong evidence of rapid and preferential liposomal interactions with PLY toxin.

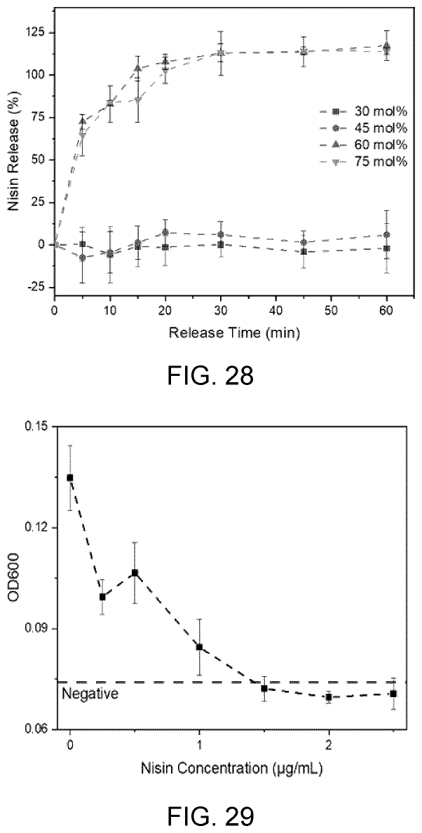

Toxin-Triggered Nisin Release. After displaying potent toxin neutralization capabilities, the ability of PLY toxin to disrupt liposomal structure and elicit nisin Z release was studied in FIG. 28.

FIG. 28 shows liposomal release of nisin Z via PLY interactions after incubation at 37° C. for a total of 1 hr at varying molar compositions of cholesterol.

It was previously found that full protection was achieved at a liposomal concentration of 10 μM for 200 ng/ml of PLY. Due to the low liposomal dosage, it was required to increase the concentration for proper detection of released nisin via CBQCA assay. As such, for release studies the liposomal dosage was increased to 200 μM and PLY was equivalently changed to 4 μg/mL to maintain the same ratio. Both a positive lysis control (0.2% Triton™X-100) and negative control (PBS) were included for all samples over the tested timepoints. The positive control for all liposomal samples showed full lysis over the entire release study, while the negative control displayed negligible nisin release over the duration of the study. As can be seen, there was little release of nisin from liposomal formulations containing both 30 and 45 mol % cholesterol, while the two cholesterol-rich formulations displayed rapid burst release of the therapeutic and full release after ~30 minutes. These findings corroborate the protection assays in that only cholesterol-rich formulations have membrane cholesterol available to facilitate binding interactions with the toxin. Beyond that, these results demonstrate the ability of the toxin to disrupt liposomal structure, likely via formation of a transmembrane pore, and cause leakage of encapsulated nisin Z in a responsive and rapid fashion. This rapid release has also been demonstrated in several studies utilizing liposomes containing fluorescent biomarkers and real-time AFM imaging [62]-[64].

Antibacterial Activity. The antibacterial efficacy of nisin was then examined through MIC measurements. Following incubation with the standardized bacterial inoculum, absorbance measurements were obtained to determine solution turbidity as can be seen in FIG. 29. Nisin showed potent inhibition of S. pneumoniae, with an MIC value of 1.5 μg/mL. Minimum bactericidal efficacy (MBC), as well as the MIC of liposomal samples of varying cholesterol content can be readily conducted, as can a time-kill assay to evaluate the kinetics of antibacterial efficacy. Testing with S. epidermidis can be readily conducted to establish the extent to which release is dependent on the presence of PLY.

FIG. 29 shows absorbance readings at 600 nm to indicate turbidity of bacterial solution with varying nisin concentration. The negative control that represents no bacterial growth has been indicated with a flat dashed line labelled “Negative”.

### Conclusion

In conclusion, a Box-Behnken response surface design successfully optimized several parameters for microfluidic development of cholesterol-rich liposomal formulations. These formulations exhibited strong loading and encapsulation of the antimicrobial peptide nisin Z while remaining uniform and monodisperse in solution. The influence of cholesterol on both particle properties and interactions with PLY were also elucidated, with key findings that a threshold cholesterol content >45 mol % was required to facilitate toxin interaction due to the interplay between cholesterol and the surrounding phospholipids present in the bilayer, both in an aqueous and film format. Beyond this, it was also determined that PLY was sufficient to destabilize the liposomal bilayer and lead to subsequent therapeutic leakage. MIC measurements and time-kill studies can be used to reveal details of antibacterial effects. Compative testing on strains that do not produce CDCs (such as S. epidermidis) can be used to study specificity. Further, studies with cells such as IMR-90 human lung fibroblasts can be used to examine cytotoxicity of the liposomes or their ability to protect against bacterial challenge. As the platform shows specificity to S. pneumoniae due to its production of the PLY and a strong safety profile to human cells, it may be an effective treatment platform, not only to counter S. pneumoniae, but also to counter various other pathogenic bacteria that also express CDC toxins. Demonstration of its utility in a dried film format, below as described in Example 3, also extends its applications into antibacterial surface coatings, devices, and diagnostics.

### Example 3

A Pneumolysin-Responsive Liposomal Coating for Selective Treatment of Streptococcus pneumoniae on a Surface

A film is prepared which is responsive to the CDC pneumolysin as released from S. pneumoniae. Pathogenesis of S. pneumoniae is largely controlled by release of the protein pneumolysin (PLY), which is part of a pore-forming toxin family of cholesterol-dependent cytolysins (CDCs).

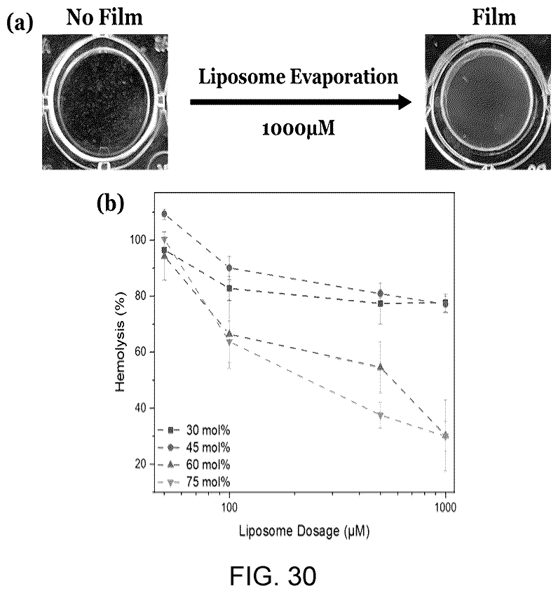

FIG. 30 illustrates liposomal films of varying molarity. Panel (a) shows that films were affixed to the bottom of a 96 well plate and fully dried (1000 μM film displayed). Following this, the protective effect of the films was examined through hemolytic assays of the treated wells at varying cholesterol content and molarity when incubated with PLY capable of induing full lysis of human erythrocytes (200 ng/ml), as shown in Panel (b).

To demonstrate the potential for use in film or coating applications, liposomal solutions were air-dried under ambient conditions and at various molarities, as seen in FIG. 30, Panel (a). This formed uniform, opaque films on the polystyrene surface and was subsequently used for sequestration studies in FIG. 30, Panel (b), under similar methodology as discussed above and a PLY concentration of 200 ng/mL.

As can be observed, the overall trend of toxin neutralization and erythrocyte protection at higher cholesterol content was maintained, with ≥60 mol % cholesterol limiting hemolytic rate due to the presence of available cholesterol for binding. It must be noted that higher liposome concentrations were required to observe a similar protective effect as the particles suspended in solution, with a hemolytic rate of ~30% at a liposomal film molarity of 1000 μM, whereas full protection was seen at 10 μM when in free-flowing in solution. This likely originates from both incomplete solubilization of the dried liposomal film, as well as changes in the lipid aggregate structure upon dehydration and subsequent rehydration that often lead to fused vesicles and an increase in size [62]. This can directly diminish the availability of surface cholesterol and hamper toxin binding. Regardless, these results indicate that liposomal films were able to retain their toxin-neutralization activity when dried onto a plastic substrate.

### Examples Only

In the preceding description, for purposes of explanation, numerous details are set forth in order to provide a thorough understanding of the embodiments. However, it will be apparent to one skilled in the art that these specific details are not required.

The embodiments described herein are intended to be examples only. Alterations, modifications and variations can be made to the particular embodiments by those of skill in the art. The scope of the claims should not be limited by the particular embodiments set forth herein, but should be construed in a manner consistent with the specification as a whole.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

### REFERENCES

All publications, patents and patent applications mentioned in this specification are indicative of the level of skill those skilled in the art to which this invention pertains and are herein incorporated by reference to the same extent as if each individual publication patent, or patent application was specifically and individually indicated to be incorporated by reference.

US 2008/0020058A1

WO 2017/049233

US 2004/0115319

US 2020/0205403

U.S. Pat. No. 11,306,186 B2

U.S. Pat. No. 10,782,238 B2

WO 2015/114666
[1] United Nations, Reducing food loss and waste: Taking Action to Transform Food Systems (Feb. 2, 2023). Available from: https://www.un.org/en/observances/end-food-waste-day
[2A] K. A. Geno, G. L. Gilbert, Y. Song, I. C. Skovsted, K. P. Klugman, C. Jones, H. B. Konradsen, M. H. Nahm, Pneumococcal Capsules and Their Types: Past, Present, and Future, Clin. Microbiol. Rev. 28 (2015) 871-899. https://doi.org/10.1128/CMR.00024-15.
[2B] A. J. Loughran, C. J. Orihuela, E. I. Tuomanen, Streptococcus pneumoniae: Invasion and Inflammation, Microbiol. Spectr. 7 (2019) 1128. https://doi.org/10.1128/microbiolspec.GPP3-0004-2018. Streptococcus.
[3] S. Mehr, N. Wood, Streptococcus pneumoniae—a review of carriage, infection, serotype replacement and vaccination, Paediatr. Respir. Rev. 13 (2012) 258-264. https://doi.org/10.1016/j.prrv.2011.12.001.
[4] J. G. Bartlett, S. F. Dowell, L. A. Mandell, T. M. File, D. M. Musher, M. J. Fine, Practice Guidelines for the Management of Community-Acquired Pneumonia in Adults, Clin. Infect. Dis. 31 (2000) 347-382.
[5] C. Troeger, Estimates of the global, regional, and national morbidity, mortality, and aetiologies of lower respiratory tract infections in 195 countries: a systematic analysis for the Global Burden of Disease Study 2015, Lancet Infect. Dis. 17 (2017) 1133-1161. https://doi.org/10.1016/S1473-3099 (17) 30396-1.
[6] B. Heras, M. J. Scanlon, J. L. Martin, Targeting virulence not viability in the search for future antibacterials, Br. J. Clin. Pharmacol. 79 (2016) 208-215. https://doi.org/10.1111/bcp.12356.
[7] L. R. K. Brooks, G. I. Mias, Streptococcus pneumoniae's Virulence and Host Immunity: Aging, Diagnostics, and Prevention, Front. I. 9 (2018) 1366. https://doi.org/10.3389/fimmu.2018.01366.
[8] A. Roth, P. Reichmann, R. Hakenbeck, The Capsule of Streptococcus pneumoniae Contributes to Virulence in the Insect Model Manduca sexta, J. Mol. Microbiol. Biotechnol. 22 (2012) 326-334. https://doi.org/10.1159/000345327.
[9] M. Domenech, E. García, M. Moscoso, Biofilm formation in Streptococcus pneumoniae, Microb. Biotechnol. 5 (2012) 455-465. https://doi.org/10.1111/j. 1751-7915.2011.00294.x.
[10] M. A. Barocchi, J. Ries, X. Zogaj, C. Hemsley, B. Albiger, A. Kanth, S. Dahlberg, J. Fernebro, M. Moschioni, V. Masignani, K. Hultenby, A. R. Taddei, K. Beiter, F. Wartha, A. Euler, A. Covacci, D. W. Holden, S. Normark, R. Rappuoli, B. Henriques-Normark, A pneumococcal pilus influences virulence and host inflammatory responses, PNAS. 103 (2006) 2857-2862. https://doi.org/10.1073/pnas.0511017103.
[11] K. K. Gosink, E. R. Mann, C. Guglielmo, E. I. Tuomanen, H. R. Masure, Role of Novel Choline Binding Proteins in Virulence of Streptococcus pneumoniae, Infect. Immun. 68 (2000) 5690-5695.
[12] N. Li, X.-Y. Yang, Z. Guo, J. Zhang, K. Cao, J. Han, G. Zhang, L. Liu, X. Sun, Q.-Y. He, Varied metal-binding properties of lipoprotein PsaA in Streptococcus pneumoniae, J. Biol. Inorg. Chem. 19 (2014) 829-838. https://doi.org/10.1007/s00775-014-1114-9.
[13] S. Manco, F. Hernon, H. Yesilkaya, J. C. Paton, P. W. Andrew, A. Kadioglu, Pneumococcal Neuraminidases A and B Both Have Essential Roles during Infection of the Respiratory Tract and Sepsis, Infect. Immun. 74 (2006) 4014-4020. https://doi.org/10.1128/IAI.01237-05.
[14] M. J. Frias, J. Melo-Cristino, M. Ramirez, The Autolysin LytA Contributes to Efficient Bacteriophage Progeny Release in Streptococcus pneumoniae, J. Bacteriol. 191 (2009) 5428-5440. https://doi.org/10.1128/JB.00477-09.
[15] W.-Y. Wholey, M. Abu-Khdeir, E. A. Yu, S. Siddiqui, O. Esimai, S. Dawid, Characterization of the Competitive Pneumocin Peptides of Streptococcus pneumoniae, Front. Cell. Infect. Microbiol. 9 (2019) 55. https://doi.org/10.3389/fcimb.2019.00055.
[16] A. T. Nishimoto, J. W. Rosch, E. I. Tuomanen, Pneumolysin: Pathogenesis and Therapeutic Target, Front. Microbiol. 11 (2020) 1543. https://doi.org/10.3389/fmicb.2020.01543.
[17] V. Martin, R. M. Bhaskara, E. Mulvihill, K. Pee, O. Yildiz, W. Kuhlbrandt, D. J. Muller, G. Hummer, Membrane perforation by the pore-forming toxin pneumolysin, PNAS. 116 (2019) 13352-13357. https://doi.org/10.1073/pnas. 1904304116.
[18] A. Marie, D. Guilmi, A. Dessen, New approaches towards the identification of antibiotic and vaccine targets in Streptococcus pneumoniae, EMBO Rep. 3 (2002) 728-734.
[19] R. A. Hirst, A. Kadioglu, C. O. Callaghan, P. W. Andrew, The role of pneumolysin in pneumococcal pneumonia and meningitis, Clin. Exp. Immunol. 138 (2004) 195-201. https://doi.org/10.1111/j. 1365-2249.2004.02611.x.
[20] L. C. Jacques, S. Panagiotou, M. Baltazar, M. Senghore, S. Khandaker, R. Xu, L. Bricio-Moreno, M. Yang, C. G. Dowson, D. B. Everett, D. R. Neill, A. Kadioglu, Increased pathogenicity of pneumococcal serotype 1 is driven by rapid autolysis and release of pneumolysin, Nat. Commun. 11 (2020) 1892. https://doi.org/10.1038/s41467-020-15751-6.
[21] R. Anderson, J. G. Nel, C. Feldman, Multifaceted Role of Pneumolysin in the Pathogenesis of Myocardial Injury in Community-Acquired Pneumonia, Int. J. Mol. Sci. 19 (2018) 1147. https://doi.org/10.3390/ijms19041147.
[22] M. V. Surve, S. Bhutda, A. Datey, A. Anil, S. Rawat, A. Pushpakaran, D. Singh, K. S. Kim, D. Chakravortty, A. Banerjee, Heterogeneity in pneumolysin expression governs the fate of Streptococcus pneumoniae during blood-brain barrier trafficking, PLOS Pathog. 14 (2018) e1007168.
[23] A. Leonard, K. Mohlis, R. Schlüter, E. Taylor, M. Lalk, K. Methling, Exploring metabolic adaptation of Streptococcus pneumoniae to antibiotics, J. Antibiot. (Tokyo). 73 (2020) 441-454. https://doi.org/10.1038/s41429-020-0296-3.
[24] J. Tang, Q. Ouyang, Y. Li, P. Zhang, W. Jin, S. Qu, F. Yang, Z. He, M. Qin, Nanomaterials for Delivering Antibiotics in the Therapy of Pneumonia, Int. J. Mol. Sci. 23 (2022) 15738.
[25] B. Chang, Y. Kinjo, M. Morita, K. Tamura, H. Watanabe, Y. Tanabe, K. Kuronuma, J. Fujita, K. Oshima, T. Maruyama, S. Abe, K. Kasahara, J. Nishi, T. Kubota, M. Ohnishi, S. Suga, K. Oishi, A. Ip, Distribution and Variation of Serotypes and Pneumococcal Surface Protein A Clades of Streptococcus pneumoniae Strains Isolated From Adult Patients With Invasive Pneumococcal Disease in Japan, Front. Cell. Infect. Microbiol. 11 (2021) 617573. https://doi.org/10.3389/fcimb.2021.617573.
[26] R. Shimbashi, M. Suzuki, B. Chang, H. Watanabe, Y. Tanabe, K. Kuronuma, K. Oshima, T. Maruyama, H. Takeda, K. Kasahara, J. Fujita, J. Nishi, T. Kubota, K. Tanaka-Taya, T. Matsui, T. Sunagawa, K. Oishi, Effectiveness of 23-Valent Pneumococcal Polysaccharide Vaccine against Invasive Pneumococcal Disease in Adults, Japan, 2013-2017, Emerg. Infect. Dis. 26 (2020) 2378-2386.
[27] G.-L. Kim, S.-H. Seon, D.-K. Rhee, Pneumonia and Streptococcus pneumoniae vaccine, Arch. Pharm. Res. 40 (2017) 885-893. https://doi.org/10.1007/s12272-017-0933-y.
[28] M. Kumar, D. K. Sarma, S. Shubham, M. Kumawat, V. Verma, P. B. Nina, D. JP, S. Kumar, B. Singh, R. R. Tiwari, Futuristic Non-antibiotic Therapies to Combat Antibiotic Resistance: A Review, Front. Microbiol. 12 (2021) 609459. https://doi.org/10.3389/fmicb.2021.609459.
[29] M. Rima, M. Rima, Z. Fajloun, J. Sabatier, B. Bechinger, T. Naas, Antimicrobial Peptides: A Potent Alternative to Antibiotics, Antibiotics. 10 (2021) 1095.
[30] J. M. Shin, J. W. Gwak, P. Kamarajan, J. C. Fenno, A. H. Rickard, Y. L. Kapila, Biomedical Applications of Nisin, J. Appl. Microbiol. 120 (2017) 1449-1465. https://doi.org/10.1111/jam. 13033.Biomedical.
[31] J. A. Majchrzykiewicz, O. P. Kuipers, J. J. E. Bijlsma, Generic and Specific Adaptive Responses of Streptococcus pneumoniae to Challenge with Three Distinct Antimicrobial Peptides, Bacitracin, LL-37, and Nisin, Antimicrob. Agents Chemother. 54 (2010) 440-451. https://doi.org/10.1128/AAC.00769-09.
[32] S. Azarmi, W. H. Roa, R. Löbenberg, Targeted delivery of nanoparticles for the treatment of lung diseases, Adv. Drug Deliv. Rev. 60 (2008) 863-875. https://doi.org/10.1016/j.addr.2007.11.006.
[33] M. Ferreira, M. Ogren, J. N. R. Dias, M. Silva, S. Gil, L. Tavares, F. Aires-da-Silva, M. M. Gaspar, S. I. Aguiar, Liposomes as Antibiotic Delivery Systems: A Promising Nanotechnological Strategy against Antimicrobial Resistance, Molecules. 26 (2021) 2047.
[34] D. Lombardo, M. A. Kiselev, Methods of Liposomes Preparation: Formation and Control Factors of Versatile Nanocarriers for Biomedical and Nanomedicine Application, Pharmaceutics. 14 (2022) 543.
[35] B. Yu, R. J. Lee, L. J. Lee, Microfluidic Methods for Production of Liposomes, Methods Enzymol. 465 (2009) 129-141. https://doi.org/10.1016/S0076-6879 (09) 65007-2.Microfluidic.
[36] M. S. Williams, K. J. Longmuir, P. Yager, A practical guide to the staggered herringbone mixer, Lab Chip. 8 (2008) 1121-1129. https://doi.org/10.1039/b802562b.A.
[37] D. Carugo, E. Bottaro, J. Owen, E. Stride, C. Nastruzzi, Liposome production by microfluidics: potential and limiting factors, Sci. Rep. 6 (2016) 25876. https://doi.org/10.1038/srep25876.
[38] H. Nsairat, D. Khater, U. Sayed, F. Odeh, A. Al Bawab, W. Alshaer, Liposomes: structure, composition, types, and clinical applications, Heliyon. 8 (2022) e09394. https://doi.org/10.1016/j.heliyon.2022.e09394.
[39] C. Webb, N. Forbes, C. B. Roces, G. Anderluzzi, G. Lou, S. Abraham, L. Ingalls, K. Marshall, T. J. Leaver, J. A. Watts, J. W. Aylott, Y. Perrie, Using microfluidics for scalable manufacturing of nanomedicines from bench to GMP: A case study using protein-loaded liposomes, Int. J. Pharm. 582 (2020) 119266. https://doi.org/10.1016/j.ijpharm.2020.119266.
[40] V. Nele, M. N. Holme, U. Kauscher, M. R. Thomas, J. J. Doutch, M. M. Stevens, Effect of Formulation Method, Lipid Composition, and PEGylation on Vesicle Lamellarity: A Small-Angle Neutron Scattering Study, Langmuir. 35 (2019) 6064-6074. https://doi.org/10.1021/acs.langmuir.8b04256.
[41] N. A. Nasab, H. H. Kumleh, M. Beygzadeh, S. Teimourian, M. Kazemzad, Delivery of curcumin by a pH-responsive chitosan mesoporous silica nanoparticles for cancer treatment, Artif. Cells, Nanomedicine, Biotechnol. 46 (2018) 75-81. https://doi.org/10.1080/21691401.2017.1290648.
[42] S. Choi, B. Kang, E. Yang, K. Kim, M. K. Kwak, P.-S. Chang, H.-S. Jung, Precise control of liposome size using characteristic time depends on solvent type and membrane properties, Sci. Rep. 13 (2023) 4728. https://doi.org/10.1038/s41598-023-31895-z.
[43] V. Joguparthi, S. Feng, B. D. Anderson, Determination of Intraliposomal pH and its Effect on Membrane Partitioning and Passive Loading of a Hydrophobic Camptothecin, DB-67, Int. J. Pharm. 352 (2009) 17-28.
[44] S. J. Wallace, J. Li, R. L. Nation, B. J. Boyd, Drug release from nanomedicines: Selection of appropriate encapsulation and release methodology, Drug Deliv. Transl. Res. 2 (2012) 284-292. https://doi.org/10.1007/s13346-012-0064-4.Drug.
[45] M.-R. Toh, G. N. C. Chiu, Liposomes as sterile preparations and limitations of sterilisation techniques in liposomal manufacturing, Asian J. Pharm. Sci. 8 (2013) 88-95. https://doi.org/10.1016/j.ajps.2013.07.011.
[46] G. F. Boafo, K. T. Magar, M. D. Ekpo, W. Qian, S. Tan, C. Chen, The Role of Cryoprotective Agents in Liposome Stabilization and Preservation, Int. J. Mol. Sci. 23 (2022) 12487.
[47] C. Han, M. Zhang, Genetic diversity and antigenicity analysis of Streptococcus pneumoniae pneumolysin isolated from children with pneumococcal infection, Int. J. Infect. Dis. 86 (2019) 57-64. https://doi.org/10.1016/j.ijid.2019.06.025.
[48] N. R. Scott, B. Mann, E. I. Tuomanen, C. J. Orihuela, Multi-Valent Protein Hybrid Pneumococcal Vaccines: A Strategy for the Next Generation of Vaccines, Vaccines. 9 (2021) 209.
[49] M. Salvador, J. L. Marques-Fernandez, A. Bunge, J. C. Martinez-Garcia, R. Turcu, D. Peddis, M. del M. Garcia-Suarez, M. D. Cima-Cabal, M. Rivas, Magnetic Nanoclusters Increase the Sensitivity of Lateral Flow Immunoassays for Protein Detection: Application to Pneumolysin as a Biomarker for Streptococcus pneumoniae, Nanomaterials. 12 (2022) 2044.
[50] L. Blanco-covián, V. Montes-garcía, A. Girard, M. T. Fernández-abedul, J. Pérez-juste, I. Pastoriza-santos, K. Faulds, D. Graham, M. C. Blanco-lópez, Au@Ag SERRS tags coupled to a lateral flow immunoassay for the sensitive detection of pneumolysin, Nanoscale. 9 (2017) 2051-2058. https://doi.org/10.1039/c6nr08432j.
[51] D. Baumgartner, S. Aebi, D. Grandgirard, S. L. Leib, A. Draeger, E. Babiychuk, L. J. Hathaway, Clinical Streptococcus pneumoniae isolates induce differing CXCL8 responses from human nasopharyngeal epithelial cells which are reduced by liposomes, BMC Microbiol. 16 (2016) 154-161. https://doi.org/10.1186/s12866-016-0777-5.
[52] R. Koffel, H. Wolfmeier, Y. Larpin, H. Besancon, R. Schoenauer, V. S. Babiychuk, P. Drucker, T. Pabst, T. J. Mitchell, E. B. Babiychuk, A. Draeger, Host-Derived Microvesicles Carrying Bacterial Pore-Forming Toxins Deliver Signals to Macrophages: A Novel Mechanism of Shaping Immune Responses, Front. Immunol. 9 (2018) 1688. https://doi.org/10.3389/fimmu.2018.01688.
[53] H. Besançon, V. Babiychuk, Y. Larpin, R. Koffel, D. Schittny, L. Brockhus, L. J. Hathaway, P. Sendi, A. Draeger, E. Babiychuk, Tailored liposomal nanotraps for the treatment of Streptococcal infections, J. Nanobiotechnology. 19 (2021) 46-61. https://doi.org/10.1186/s12951-021-00775-x.
[54] P. Drücker, S. Bachler, H. Wolfmeier, R. Schoenauer, R. Köffel, V. S. Babiychuk, P. S. Dittrich, A. Draeger, E. B. Babiychuk, Pneumolysin-damaged cells benefit from non-homogeneous toxin binding to cholesterol-rich membrane domains, BBA-Mol. Cell Biol. Lipids. 1863 (2018) 795-805. https://doi.org/10.1016/j.bbalip.2018.04.010.
[55] B. D. Henry, D. R. Neill, K. A. Becker, S. Gore, L. Bricio-moreno, R. Ziobro, M. J. Edwards, K. Mühlemann, J. Steinmann, B. Kleuser, L. Japtok, M. Luginbühl, H. Wolfmeier, A. Scherag, E. Gulbins, A. Kadioglu, A. Draeger, E. B. Babiychuk, Engineered liposomes sequester bacterial exotoxins and protect from severe invasive infections in mice, Nat. Biotechnol. 33 (2015) 81-88. https://doi.org/10.1038/nbt.3037.
[56] P.-F. Laterre, G. Colin, P.-F. Dequin, T. Dugernier, T. Boulain, S. A. da Silveira, F. Lajaunias, A. Perez, B. François, CAL02, a novel antitoxin liposomal agent, in severe pneumococcal pneumonia: a first-in-human, double-blind, placebo-controlled, randomized trial, Lancet Infect. Dis. 19 (2019) 620-630. https://doi.org/10.1016/S1473-3099 (18) 30805-3.
[57] L. Lin, X. Zhang, C. Zhao, H. Cui, Liposome containing nutmeg oil as the targeted preservative against Listeria monocytogenes in dumplings, RSC Adv. 6 (2016) 978-986. https://doi.org/10.1039/c5ra22098j.
[58] H. Y. Cui, J. Wu, L. Lin, Inhibitory effect of liposome-entrapped lemongrass oil on the growth of Listeria monocytogenes in cheese, J. Dairy Sci. 99 (2016) 6097-6104. https://doi.org/10.3168/jds.2016-11133.
[59] S. J. Billington, B. H. Jost, J. G. Songer, Thiol-activated cytolysins: Structure, function and role in pathogenesis, FEMS Microbiol. Lett. 182 (2000) 195-205. https://doi.org/10.1016/S0378-1097 (99) 00536-4.
[60] M. Pleckaityte, Cholesterol-Dependent Cytolysins Produced by Vaginal Bacteria: Certainties and Controversies, Front. Cell. Infect. Microbiol. 9 (2020) 452. https://doi.org/10.3389/fcimb.2019.00452.
[61] J. J. Flanagan, R. K. Tweten, A. E. Johnson, A. P. Heuck, Cholesterol Exposure at the Membrane Surface Is Necessary and Sufficient to Trigger Perfringolysin O Binding, Biochemistry. 48 (2009) 3977-3987. https://doi.org/10.1021/bi9002309.Cholesterol.
[62] J. C. Evans, B. A. Johnstone, S. L. Lawrence, C. J. Morton, M. P. Christie, M. W. Parker, R. K. Tweten, A Key Motif in the Cholesterol-Dependent Cytolysins Reveals a Large Family of Related Proteins, MBio. 11 (2020) e02351-20. https://doi.org/10.1128/mBio.02351-20.
[63] A. P. Heuck, R. K. Tweten, A. E. Johnson, Assembly and Topography of the Prepore Complex in Cholesterol-Dependent Cytolysins, J. Biol. Chem. 278 (2003) 31218-31225. https://doi.org/10.1074/jbc.M303151200.
[64] C. Leung, N. V Dudkina, N. Lukoyanova, A. W. Hodel, I. Farabella, A. P. Pandurangan, N. Jahan, M. P. Damaso, D. Osmanovi, C. F. Reboul, M. A. Dunstone, P. W. Andrew, R. Lonnen, M. Topf, H. R. Saibil, B. W. Hoogenboom, Stepwise visualization of membrane pore formation by suilysin, a bacterial cholesterol-dependent cytolysin, Elife. 3 (2014) e04247. https://doi.org/10.7554/eLife.04247.
[65] Food Packaging Market Size & Trends in “Food Packaging Market Size, Share & Trends Analysis Report by Type, By Material, By Application, By Region, And Segment Forecasts, 2023-2030. (2023) Available from: https://www.grandviewresearch.com/industry-analysis/food-packaging-market
[66] Walczak-Nowicka, Herbet M. Sodium Benzoate-Harmfulness and Potential Use in Therapies for Disorders Related to the Nervous System: A Review. Nutrients. 2022; 14:1497.
[67] Butylated Hydroxyanisole CAS No. 25013-16-5, Report on Carcinogens, Fifteenth Edition, 2021, RoC Profile p. 1-2, https://ntp.niehs.nih.gov/ntp/roc
[68] Fu Y, Dudley E G. Antimicrobial-coated films as food packaging: A review. Comprehensive Reviews in Food Science and Food Safety. 2021; 20:3404-37.
[69] Müller P, Schmid M. Intelligent Packaging in the Food Sector: A Brief Overview. Foods. 2019; 8:16.
[70] Tweten R K. Cholesterol-Dependent Cytolysins, a Family of Versatile Pore-Forming Toxins. Infect Immun. 2005; 73:6199-209.
Gelber S E, Aguilar J L, Lewis K L T, Ratner A J. Functional and phylogenetic characterization of Vaginolysin, the human-specific cytolysin from Gardnerella vaginalis. J Bacteriol. 2008; 190:3896-903.

1. Bacteria-responsive particles encapsulating a releasable agent for interaction with a target bacteria that releases a cholesterol-dependent cytolysin (CDC), said particles comprising:
surface-exposed cholesterol, and
said releasable agent encapsulated within the particles, for interaction with the target bacteria upon release,
wherein interaction of the surface-exposed cholesterol of the particles with the CDC released from the target bacteria causes particle porosity and release of the agent from the particles.

2. The particles of claim 1, wherein when the CDC binds to the surface-exposed cholesterol on the surface of the particles, the particle porosity is from pores forming in the surface of the particles, rendering the particles permeable to release the agent through the pores so formed.

3. The particles of claim 2, wherein surface of the particles comprises a lipid bilayer or a phospholipid layer.

4. The particles of claim 1,
wherein the particles comprise liposomes, nanoparticles, lipid nanoparticles, polymeric nanoparticles or combinations thereof;
wherein the particles are cholesterol-rich, comprising from 30-80 mol % cholesterol, from 55-75 mol % cholesterol, or 60-70 mol % cholesterol;
and/or
wherein the particles additionally comprise egg phosphatidylcholine (EPC), egg phosphatidylglycerol-sodium (EPG-Na), propylene glycol, a natural phospholipid sourced from egg or soybean, a synthetic phospholipid, a neutral phospholipid, an anionic phospholipid, a cationic phospholipid, a surface ligand, and/or an ionizable lipid.

5. (canceled)

6. (canceled)

7. (canceled)

8. The particles of claim 1, additionally comprising:
Phosphatidylcholine (PC);
Phosphatidylethanolamine (PE);
Phosphatidylserine (PS);
Phosphatidylglycerol (PG);
Phosphatidic acid (PA);
Phosphatidylinositol (PI);
Phosphatidylethanolamine (DPPE);
Dipalmitoylphosphatidylcholine (DPPC);
1,2-distearoyl-sn-glycero-3-phosphoethanolamine (DSPE);
1,2-distearoyl-sn-glycero-3-phosphocholine (DSPC);
1,2-Dioleoylphosphatidylethanolamine (DOPE);
1,2-Dioleoyl-sn-glycero-3-phosphocholine (DOPC);
Hydrogenated Soybean Phosphatidylcholine (HSPC);
1-palmitoyl-2-oleoyl-sn-glycero-3-phosphocholine (POPC);
1,2-Dimyristoyl-sn-glycero-3-phosphocholine (DMPC);
1,2-Dipalmitoyl-sn-glycero-3-phosphatidic acid sodium salt (DPPA);
Dipalmitoylphosphatidylglycerol (DPPG);
1,2-Distearoyl-sn-glycero-3-phosphate (DSPA);
1,2-Distearoyl-sn-glycero-3-phosphoglycerol (DSPG);
1,2-dioleoyl-3-trimethylammonium-propane (DOTAP);
1,2-di-O-octadecenyl-3-trimethylammonium propane (DOTMA);
DC-cholesterol (hydrochloride) (DC-Chol);
Dimethyldioctadecylammonium (bromide) (DDAB);
Polyethylene oxide (PEG);
Hyaluronic acid (HA);
Polyvinyl acetate (PVA);
Chitosan;
a protein;
an antibody;
an aptamer;
(6Z,9Z,28Z,31Z)-Heptatriaconta-6,9,28,31-tetraen-19-yl 4-(dimethylamino)butanoate (DLin-MC3-DMA);
1,2-dioleoyl-3-dimethylammonium-propane (DODAP);
[(4-hydroxybutyl)azanediyl]di(hexane-6,1-diyl)bis(2-hexyldecanoate) (ALC-0315);
9-Heptadecanyl 8-{(2-hydroxyethyl) [6-oxo-6-(undecyloxy) hexyl]amino}octanoate (SM-102);
1,2-dioleyloxy-3-dimethylaminopropane (DODMA);
N-(4-carboxybenzyl)-N,N-dimethyl-2,3-bis(oleoyloxy) propan-1-aminium (DOBAQ);
1-methyl-4,4-bis[(9Z,12Z)-9,12-octadecadien-1-yloxy]-piperidine (YSK05);
N,N-dimethyl-2,3-bis[(9Z,12Z)-9,12-octadecadien-1-yloxy]-1-propanamine (DLin-DMA);
N,N-dimethyl-2,2-di-(9Z,12Z)-9,12-octadecadien-1-yl-1,3-dioxolane-4-ethanamine (DLin-KC2-DMA);
5-(dimethylamino)-pentanoic acid, (6Z)-1,2-di-(4Z)-4-decen-1-yl-6-dodecen-1-yl ester (CL1);
1,2-Distearoyl-sn-Glycero-3-Phosphatidylglycerol sodium (5A2-SC8); or
a combination thereof.

9. The particles of claim 1, wherein the releasable agent is an antibacterial agent, an indicator, a preservative, a bactericide, a broad spectrum bactericide, a fungicide, a virucide, an antimicrobial organic acid, a bacteriocin, a protein toxin, an antimicrobial peptide, an enzyme, an essential oil with bactericidal activity, a plant extract, a metal ion, an oxidizer, an antioxidant, or a combination thereof; and/or
wherein the releasable agent is a food-grade, cosmetic-grade, or pharmaceutical-grade agent.

10. (canceled)

11. The particles of claim 9, wherein the releasable agent is an antimicrobial peptide (AMP) present in the particles in an amount of from 10 to 200 μg/mL, or from 50 to 100 μg/mL.

12. (canceled)

13. The particles of claim 1, having an average diameter of from 50 to 1000 nm, an average diameter of from 100 nm to 500 nm, an average diameter of from 300 to 400 nm, or having an average diameter of about 300 nm;
wherein the size distribution of the particles is represented by a polydispersity index (PDI) value of from about 0.2 to 0.3, or from 0.25 to 0.28;
and/or
wherein the particles have a zeta potential of from −20 to −80 mV, from −25 to −70, or from −40 to −60 mV to render the particles stable through electrostatic repulsion.

14. (canceled)

15. (canceled)

16. The particles of claim 1, wherein the CDC and target bacteria are selected from the group consisting of:
tetanolysin from C. tetani;
botulinolysin from C. botulinum;
perfringolysin O from C. perfringens;
septicolysin O from C. septicum;
histolyticolysin O from C. histolyticum;
novyilysin from C. Novyi A (oedematiens);
chauveolysin from C. chauvoei;
bifermentolysin from C. bifermentans;
sordellilysin from C. sordelli;
pyolysin from A. pyogenes;
laterosporolysin from B. laterosporus;
vaginolysin from G. vaginalis;
inerolysin from L. iners;
aveolysin from P. alvei;
lectinolysin from S. mitis;
intermedilysin from S. intermedius;
suilysin from S. suis;
pneumolysin from S. pneumoniae;
streptolysin O from S. canis;
streptolysin O from S. Dysgalactiae subsp. Equisimilis;
streptolysin O from S. pyogenes;
cereolysin O from B. cereus;
thuringolysin O from B. thuringiensis;
anthrolysin O from B. anthracis;
seeligerolysin O from L. seeligeri;
ivanolysin O from L. ivanovii; and
listeriolysin O from L. monocytogenes.

17. A film, a coating, a food packaging container or food packaging insert comprising the bacteria-responsive particles of claim 1.

18. (canceled)

19. A substrate or surface comprising the bacteria-responsive particles of claim 1 applied thereto, wherein said substrate or surface comprises packaging for a food, cosmetic, pharmaceutical, hygiene product packaging, a medical device, or a substrate or surface susceptible to growth of, or exposure to, the target bacteria.

20. A method of preparing bacteria-responsive particles encapsulating a releasable agent for interaction with a target bacteria, when the target bacteria releases a cholesterol-dependent cytolysin (CDC), the method comprising:
preparing a particle-forming composition for providing particles with a lipid-rich total cholesterol content of from 30-85 mol %, from 55-75 mol %, or from 60-70 mol %, said composition comprising the releasable agent;
sonicating the composition for 80 s to 600 s, such as about 200 s;
forming particles having an average diameter of from 50 to 1000 nm, an average diameter of from 100 nm to 500 nm, an average diameter of from 300 to 400 nm, or having an average diameter of about 300 nm;
filtering the particles to exclude sizes over about 35 kDa, over about 30 kDa, or over about 20 kDa; and
washing the particles to remove non-encapsulated agent;
wherein the prepared particles comprise surface-exposed cholesterol, and the releasable agent is encapsuled within said particles.

21. The method of claim 20,
wherein the particles comprise liposomes, nanoparticles, lipid nanoparticles, polymeric nanoparticles or combinations thereof;
wherein egg L-α-phosphatidylglycerol (EPG) and L-α-phosphatidylcholine (EPC) are included in the particle-forming composition; and/or
wherein a natural phospholipid sourced from egg or soybean, a synthetic phospholipid, a neutral phospholipid, an anionic phospholipid, a cationic phospholipid, a surface ligand, an ionizable lipid, or a combination thereof is included in the particle-forming composition.

22. (canceled)

23. (canceled)

24. The method of claim 20, wherein one or a combination of the following is included in the particle forming composition:
Phosphatidylcholine (PC);
Phosphatidylethanolamine (PE);
Phosphatidylserine (PS);
Phosphatidylglycerol (PG);
Phosphatidic acid (PA);
Phosphatidylinositol (PI);
Phosphatidylethanolamine (DPPE);
Dipalmitoylphosphatidylcholine (DPPC);
1,2-distearoyl-sn-glycero-3-phosphoethanolamine (DSPE);
1,2-distearoyl-sn-glycero-3-phosphocholine (DSPC);
1,2-Dioleoylphosphatidylethanolamine (DOPE);
1,2-Dioleoyl-sn-glycero-3-phosphocholine (DOPC);
Hydrogenated Soybean Phosphatidylcholine (HSPC);
1-palmitoyl-2-oleoyl-sn-glycero-3-phosphocholine (POPC);
1,2-Dimyristoyl-sn-glycero-3-phosphocholine (DMPC);
1,2-Dipalmitoyl-sn-glycero-3-phosphatidic acid sodium salt (DPPA);
Dipalmitoylphosphatidylglycerol (DPPG);
1,2-Distearoyl-sn-glycero-3-phosphate (DSPA);
1,2-Distearoyl-sn-glycero-3-phosphoglycerol (DSPG);
1,2-dioleoyl-3-trimethylammonium-propane (DOTAP);
1,2-di-O-octadecenyl-3-trimethylammonium propane (DOTMA);
DC-cholesterol (hydrochloride) (DC-Chol);
Dimethyldioctadecylammonium (bromide) (DDAB);
Polyethylene oxide (PEG);
Hyaluronic acid (HA);
Polyvinyl acetate (PVA);
Chitosan;
a protein;
an antibody;
an aptamer;
(6Z,9Z,28Z,31Z)-Heptatriaconta-6,9,28,31-tetraen-19-yl 4-(dimethylamino)butanoate (DLin-MC3-DMA);
1,2-dioleoyl-3-dimethylammonium-propane (DODAP);
[(4-hydroxybutyl)azanediyl]di(hexane-6,1-diyl)bis(2-hexyldecanoate) (ALC-0315);
9-Heptadecanyl 8-{(2-hydroxyethyl) [6-oxo-6-(undecyloxy) hexyl]amino}octanoate (SM-102);
1,2-dioleyloxy-3-dimethylaminopropane (DODMA);
N-(4-carboxybenzyl)-N,N-dimethyl-2,3-bis(oleoyloxy) propan-1-aminium (DOBAQ);
1-methyl-4,4-bis[(9Z,12Z)-9,12-octadecadien-1-yloxy]-piperidine (YSK05);
N,N-dimethyl-2,3-bis[(9Z,12Z)-9,12-octadecadien-1-yloxy]-1-propanamine (DLin-DMA);
N,N-dimethyl-2,2-di-(9Z,12Z)-9,12-octadecadien-1-yl-1,3-dioxolane-4-ethanamine (DLin-KC2-DMA);
5-(dimethylamino)-pentanoic acid, (6Z)-1,2-di-(4Z)-4-decen-1-yl-6-dodecen-1-yl ester (CL1); or
1,2-Distearoyl-sn-Glycero-3-Phosphatidylglycerol sodium (5A2-SC8).

25. The method of claim 20, wherein the releasable agent is an antibacterial agent, an indicator, a preservative, a bactericide, a broad spectrum bactericide, a fungicide, a virucide, an antimicrobial organic acid, a bacteriocin, a protein toxin, an antimicrobial peptide, an enzyme, an essential oil with bactericidal activity, a plant extract, a metal ion, an oxidizer, an antioxidant, or a combination thereof.

26. The method of claim 20, wherein the particle-forming composition is prepared using a microfluidic system comprising an aqueous stream and an ethanolic stream having flow rate ratio between the aqueous and ethanolic streams of from 2:1 to 5:1, or a flow rate ratio of about 3:1.

27. The method of claim 24, wherein cholesterol, egg L-α-phosphatidylglycerol (EPG), and L-α-phosphatidylcholine (EPC) are present in the ethanolic stream.

28. The method according to claim 26, claim 26 or 27, wherein the aqueous stream comprises the releasable agent.

29. (canceled)

30. A method of inhibiting a target bacteria comprising exposure of said target bacteria to the bacteria-responsive particles of claim 1.
